(19) United States
(12) Patent Application Publication
(10) Pub. No.: US 2003/0207846 A1
(43) Pub. Date: Nov. 6, 2003

(54) Treatment of pain, inflammation, and inflammation-related disorders with a combination of a cyclooxygenase-2 selective inhibitor and aspirin

(75) Inventor: Stephen P. MacMillan, Newtown, PA (US)

Correspondence Address: Charles E. Dunlap, Keenan Building, Third Floor, 1330 Lady Street, Columbia, SC, 29201, US

(73) Assignee: Pharmacia Corporation, St. Louis, MO

(21) Appl. No.: 10/337,760

(22) Filed: Jan. 7, 2003

Related U.S. Application Data
(60) Provisional application No. 60/346,560, filed on Jan. 7, 2002.

(51) Int. Cl. A61K 31/4184; A61K 31/415

(52) U.S. Cl. 514/161; 514/394; 514/406

(57) ABSTRACT

A method for the prevention, treatment, or amelioration of pain, inflammation, or inflammation-related disorder in a subject that is in need of such prevention, treatment or amelioration, involves the administration to the subject of a cyclooxygenase-2 selective inhibitor or prodrug thereof and enteric coated aspirin. A method can also involve the administration of a cyclooxygenase-2 selective inhibitor and aspirin in an amount lower than 75 mg/day. A method can also involve the administration of a cyclooxygenase-2 selective inhibitor and aspirin where the cycloxygenase-2 selective inhibitor is BMS-347070, S-33516, CS-502, darbufelone, LAS 34475, LAS 34556, L-745337, SD-8381, RWJ-63556, L-784512, COX-189, ABT-963, or valdecoxib, or any pharmaceutical salt or prodrug thereof. Compositions, pharmaceutical compositions and kits that can be used with the methods are also described.

### CROSS-REFERENCE TO RELATED APPLICATIONS

This application is related to, and claims priority to, U.S. Provisional Patent Application Serial No. 60/346,560, filed Jan. 7, 2002, which is hereby incorporated by reference herein in its entirety; and is also related to a patent application having the same assignee and having the title: Drug Mixture With Enhanced Dissolution Rate, which was filed Jan. 7, 2003.

### BACKGROUND OF THE INVENTION

(1) Field of the Invention:

The present invention relates to methods and compositions for the prevention, treatment and amelioration of pain, inflammation and inflammation-related disorders, and more particularly to methods and compositions for the prevention, treatment and amelioration of pain, inflammation and inflammation-related disorders that involve a combination of a cyclooxygenase-2 selective inhibitor and aspirin.

(2) Description of the Related Art:

Inflammation is a manifestation of the body's response to tissue damage and infection. Although the complex mechanisms of inflammation are not fully elucidated, inflammation is known to have a close relationship with the immune response and to be associated with pain and fever in the subject.

Prostaglandins are known to be important mediators of inflammation, as well as to regulate other significant non-inflammation-related functions. Regulation of the production and activity of prostaglandins has been a common target of antiinflammatory drug discovery activities. However, common non-steroidal antiinflammatory drugs (NSAIDs) that are active in reducing the prostaglandin-induced pain and swelling associated with the inflammation process also have an effect, sometimes adverse, upon other prostaglandin-regulated processes not associated with the inflammation process. Moreover, the use of high doses of many common NSAIDs can produce severe side effects that limit their therapeutic potential.

The mechanism ascribed to many of the common NSAIDs is the modulation of prostaglandin synthesis by inhibition of cyclooxygenases that catalyze the transformation of arachidonic acid—the first step in the prostaglandin synthesis pathway. It has recently been discovered that two cyclooxygenases are involved in this transformation. These enzymes have been termed cyclooxygenase-1 (Cox-1) and cyclooxygenase-2 (Cox-2). See, Needleman, P. et al., J. Rheumatol., 24, Suppl.49:6-8 (1997). See, Fu, J. Y., et al., J. Biol. Chem., 265(28):16737-40 (1990).

Cox-1 has been shown to be a constitutively produced enzyme that is involved in many of the non-inflammatory regulatory functions associated with prostaglandins. Cox-2, on the other hand, is an inducible enzyme having significant involvement in the inflammatory process. Many of the common NSAIDs are now known to be inhibitors of both Cox-1 and Cox-2. Accordingly, when administered in sufficiently high levels, these NSAIDs affect not only the inflammatory consequences of Cox-2 activity, but also the beneficial activities of Cox-1.

Recently, compounds have been discovered that selectively inhibit the activity of Cox-2 to a much greater extent than the activity of Cox-1. The Cox-2-selective inhibitors are believed to offer advantages that include the capacity to prevent or reduce inflammation while avoiding harmful side effects associated with the inhibition of Cox-1. Several Cox-2 selective inhibitors are now commercially available, and celecoxib is available under the trade name Celebrex® (Pharmacia Corporation), and rofecoxib is available under the trade name Vioxx® (Merck & Co.).

As mentioned above, certain NSAIDs—aspirin in particular—are known to cause undesirable side effects in some users. These side effects can include upper GI tract complications, such as bleeding or perforation. Accordingly, the co-administration of aspirin with a Cox-2 selective inhibitor would appear to be counterintuitive, because one would not wish to jeopardize the reduced upper GI tract complications offered by the Cox-2 selective inhibitor by adding a known GI tract irritant—such as aspirin—that offers similar anti-inflammatory and analgesic effects to those provided by the Cox-2 inhibitor.

However, in addition to its anti-inflammatory and analgesic effects, aspirin has been reported to provide certain cardioprotective benefits, at least when administered at conventional dosage levels—about one 325 mg tablet per day. Attempts to avoid the undesirable GI tract complications described above while maintaining the beneficial cardioprotective effects of aspirin have included the use of aspirin at lower-than-normal dosage rates (low-dose), or the use of aspirin having a coating that modulates the contact of aspirin with the stomach lining. The efficacy of each of these methods in obtaining the desired benefits while avoiding the undesirable side effects, however, remains somewhat unclear.

Budd et al., J. R. Soc. Med., 86(5):261-261 (1993) reported that aspirin preparations at 100-300 mg/day were very effective as an antiplatelet agent, whether plain or in a slow release form. However, Weber et al, Thromb. Res., 97(5):365-367 (2000) concluded that 40 mg of aspirin were not sufficient to inhibit platelet function under conditions of limited compliance.

Furthermore, it seems to remain unclear whether coated aspirin, especially at low dose, provides a benefit over non-coated aspirin in terms of GI tract irritation, bleeding, etc. For example, de Abajo et al., BMC Clin. Pharmacol., 1(1):1 (2001), tested the risk of upper gastrointestinal bleeding and perforation associated with low-dose aspirin (75-300 mg/day) as plain and enteric coated formulations. They concluded that low-dose aspirin increased the risk of upper GI tract complications and that a coating did not modify that effect. They also reported that concomitant use of low-dose aspirin and NSAIDs (such as paracetamol, steroids, anticoagulants, selective serotonin reuptake inhibitors and antiulcer drugs) at high doses put patients at a higher risk of upper GI tract complications.

On the other hand, Savon et al., in Am. J. Gastroenterol., 90(4):581-585 (1995), reported that enteric coated aspirin administered at 325 mg/day caused significantly lower gastrointestinal blood loss than plain aspirin at the same dosage level. Others have reported that enteric coated aspirin at 300 mg/day is less gastrotoxic than regular aspirin (Blondon et al., Fundam. Clin. Pharmacol., 14(2):155-157 (2000)); and, that enteric coated aspirin at 100 mg/day caused less gastroduodenal damage over 7 days than the same dose of plain aspirin. Dammann et al., Aliment. Pharmacol. Ther., 13(8):1109-1114 (1999).

Few reports have been found that describe the co-administration of aspirin with a Cox-2 selective inhibitor. The reports that were found apparently focus on elucidating the effect of a Cox-2 selective inhibitor when co-administered with aspirin on the anti-platelet aggregation activity, or the cardioprotective activity, of the aspirin.

In one instance, Greenberg, H. E. et al., J. Clinical Pharmacology, 40(12 Pt. 2):1509-1515 (2000), reported that rofecoxib (50 mg/day) administered in combination with aspirin (81 mg/day) to healthy human subjects did not alter the anti-platelet effects of the aspirin (inhibition of platelet aggregation and ex-vivo serum-generated thromboxane B2 production), and was well-tolerated when administered alone or in combination with the aspirin.

In U.S. Pat. No. 6,136,804 to Nichtberger, a pharmaceutical composition and a method for treating, preventing, or reducing the risk of developing a condition selected from acute coronary ischemic syndrome, thrombosis, thromboembolism, thrombotic occlusion and reocclusion, restenosis, transient ischemic attack, and first or subsequent thrombotic stroke, in a patient, by administering an antiplatelet agent in combination with a cyclooxygenase-2 inhibitor are described. Aspirin is identified as a suitable antiplatelet agent, and dosage amounts of aspirin of from about 75 mg/day to about 325 mg/day are discussed.

Given the present understanding of the beneficial and detrimental effects of aspirin and the cycloxygenase-2 selective inhibitors, it would be useful to provide a method for treating, preventing or alleviating pain, inflammation, and inflammation-related disorders that would avoid or reduce the GI-tract complications that are commonly associated with the use of aspirin, while obtaining the benefit of aspirin's cardioprotective benefits. It would also be useful if such benefits could be provided at dosage levels that are lower than would normally be associated with such benefits.

### SUMMARY OF THE INVENTION

Briefly, therefore the present invention is directed to a novel method for the prevention, treatment, or amelioration of pain, inflammation, or inflammation-related disorder in a subject that is in need of such prevention, treatment or amelioration, the method comprising administering to the subject a cyclooxygenase-2 selective inhibitor or prodrug thereof and enteric coated aspirin.

The present invention is also directed to a novel composition for the treatment, prevention, or inhibition or pain, inflammation, or inflammation-associated disorder comprising enteric coated aspirin and a cyclooxygenase-2 selective inhibitor or prodrug thereof.

The present invention is also directed to a novel pharmaceutical composition comprising enteric coated aspirin; a cyclooxygenase-2 selective inhibitor or prodrug thereof; and a pharmaceutically-acceptable excipient.

The present invention is also directed to a novel kit that is suitable for use in the treatment, prevention or inhibition of pain, inflammation or inflammation-associated disorder, the kit comprises a first dosage form comprising enteric coated aspirin and a second dosage form comprising a cyclooxygenase-2 selective inhibitor or prodrug thereof, in quantities which comprise a therapeutically effective amount of the combination of the compounds for the treatment, prevention, or inhibition of pain, inflammation or inflammation-associated disorder.

The present invention is also directed to a novel method for the prevention, treatment, or amelioration of pain, inflammation, or inflammation-related disorder in a subject that is in need of such prevention, treatment or amelioration, the method comprising administering to the subject a cyclooxygenase-2 selective inhibitor or prodrug thereof and a low-dose of aspirin, wherein the aspirin is administered at a dosage level of below 75 mg/day.

The present invention is also directed to a novel composition comprising a cyclooxygenase-2 selective inhibitor or prodrug thereof and a low-dose of aspirin, wherein the aspirin is present in an amount of below 75 mg.

The present invention is also directed to a novel pharmaceutical composition comprising a cyclooxygenase-2 selective inhibitor and a low-dose of aspirin in combination with a pharmaceutically acceptable carrier, wherein the aspirin is present in an amount of below 75 mg.

The present invention is also directed to a novel kit that is suitable for use in the treatment, prevention or inhibition of pain, inflammation or inflammation-associated disorder, the kit comprises a first dosage form comprising less than 75 mg of aspirin and a second dosage form comprising a cyclooxygenase-2 selective inhibitor or prodrug thereof, wherein the cyclooxygenase-2 selective inhibitor is present in a quantity which, along with the quantity of aspirin, comprises a therapeutically effective amount of the combination of the compounds for the treatment, prevention, or inhibition of pain, inflammation or inflammation-associated disorder.

The present invention is also directed to a novel method for the prevention, treatment, or amelioration of pain, inflammation, or inflammation-related disorder in a subject that is in need of such prevention, treatment or amelioration, the method comprising administering to the subject a combination comprising cyclooxygenase-2 selective inhibitor and aspirin, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof.

The present invention is also directed to a novel composition comprising a cyclooxygenase-2 selective inhibitor and aspirin, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof.

The present invention is also directed to a novel pharmaceutical composition comprising a cyclooxygenase-2 selective inhibitor and aspirin in combination with a pharmaceutically acceptable carrier, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof.

The present invention is also directed to a novel kit that is suitable for use in the treatment, prevention or inhibition of pain, inflammation or inflammation-associated disorder, the kit comprises a first dosage form comprising aspirin and a second dosage form comprising a cyclooxygenase-2 selective inhibitor or prodrug thereof, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof, and wherein the aspirin and the cyclooxygenase-2 selective inhibitor are present in quantities which comprise a therapeutically effective amount of the combination of the compounds for the treatment, prevention, or inhibition of pain, inflammation or inflammation-associated disorder.

Among the several advantages found to be achieved by the present invention, therefore, may be noted the provision of a method for treating, preventing or alleviating pain, inflammation, and inflammation-related disorders that avoids or reduces the GI-tract complications that are commonly associated with the use of aspirin, while obtaining the benefit of aspirin's cardioprotective benefits, and the provision of a method that provides such benefits at dosage levels that are lower than would normally be associated with such benefits, and the provision of compositions, pharmaceutical compositions and kits that are useful for the use of the present method.

### DETAILED DESCRIPTION OF THE PREFERRED EMBODIMENTS

In accordance with the present invention, it has been discovered that pain, inflammation and inflammation-related disorders can be effectively prevented, treated, and/or ameliorated in subjects that are in need of such prevention, treatment or amelioration by administering to the subject a cyclooxygenase-2 selective inhibitor or prodrug thereof and enteric coated aspirin. In another embodiment, the method can be effected by administering to the subject a cyclooxygenase-2 selective inhibitor or prodrug thereof and a low-dose of aspirin, wherein the aspirin is administered at a dosage level of below 75 mg/day. In yet another embodiment, the prevention, treatment and/or amelioration can be effected by administering to the subject a combination comprising cyclooxygenase-2 selective inhibitor and aspirin, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof.

These and other embodiments of the novel method provide a safe and efficacious technique for preventing, treating and alleviating pain and inflammation and disorders that are associated with inflammation. In addition, the novel method is believed to provide desirable cardioprotective benefits.

It is well known that cyclooxygenase-2 selective inhibitors provide the analgesic benefits of NSAIDS and avoid upper GI-tract irritation/erosion that is common to the use of aspirin. On the other hand, it is believed that aspirin provides cardioprotective benefits that may not be provided by cyclooxygenase-2 selective inhibitors. However, the co-administration of aspirin along with a cycloxygenase-2 selective inhibitor is counterintuitive because aspirin is a cause of a problem that the use of cyclooxygenase-2 selective inhibitors is designed to avoid—namely upper GI-tract irritation. These problems have, in fact, been recognized in the recent literature, but the proposed solution was the discovery of a hypothetical new drug that combined the pharmacokinetic characteristics of aspirin-like drugs with the selectivity of a cycloxygenase-2 inhibitor such as rofecoxib. See, Brune et al, Clin. Exp. Rheumatol., 19(6 Suppl 25):S51-S57 (2001). However, the present innovative methods of the co-administration of aspirin along with a cyclooxygenase-2 selective inhibitor that are disclosed herein avoid the problem of upper GI-tract irritation, but also provide the benefits offered by both medications, and without the labor, expense and uncertainty required for the discovery of a new drug. In particular, the present method permits the effective treatment, prevention and amelioration of pain, inflammation, and inflammation-related disorders and also the cardioprotective benefits of aspirin, while decreasing, and preferably avoiding, upper GI-tract irritation.

In addition to being an efficacious method and composition for treating, preventing and/or ameliorating such disorders in a subject, it is believed that such method and composition can also provide desirable properties such as stability, ease of handling, ease of compounding, lack of side effects, ease of preparation or administration, and the like.

The novel method and compositions comprise the use of aspirin and a cyclooxygenase-2 selective inhibitor in combination. As the term is used herein, “aspirin”, means 2-(acetyloxy)benzoic acid, having a CAS RN of 50-78-2, and which can also be referred to as salicylic acid acetate and acetylsalicylic acid, among other names. Aspirin that is useful in the present invention can be from any source and can be of any purity that is commonly known in the trade to be acceptable for pharmaceutical use. As used herein, the term “aspirin” should be understood to include any prodrug, any pharmaceutically acceptable salt, and any derivative of aspirin, which is capable of providing or producing aspirin when exposed to normal physiological conditions such as occur in the bloodstream or GI tract of a mammal.

“Enteric coated aspirin”, as those terms are used herein, refer to aspirin in a form that is capable of delaying the release of the aspirin for absorption after oral ingestion by a subject until after the aspirin has passed, at least partially, into the lower gastrointestinal tract of the subject. It is preferred that the enteric coated aspirin delay such release of aspirin until after the aspirin has passed predominantly into the lower GI tract of the subject.

Methods for the preparation of enteric coated aspirin are well-known in the art, and a description of various forms of enteric coated aspirin and methods for their preparation can be found in U.S. Pat. Nos. 5,238,686; 4,975,283; 4,900,559; 4,857,337; 4,780,318; 4,507,276; and 4,443,497; among others.

Another component of the combination of the present invention is a cycloxygenase-2 selective inhibitor. The terms “cyclooxygenase-2 selective inhibitor”, or “Cox-2 selective inhibitor”, which can be used interchangeably herein, embrace compounds which selectively inhibit cyclooxygenase-2 over cyclooxygenase-1, and also include pharmaceutically acceptable salts of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity. Preferred cyclooxygenase-2 selective inhibitors of the present invention have a cyclooxygenase-2 IC₅₀ of less than about 1 μM, more preferred of less than about 0.5 μM, and even more preferred of less than about 0.2 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic cyclooxygenase-2 selective inhibitor valdecoxib. An example of a preferred Cox-2 selective inhibitor prodrug is parecoxib sodium. A class of prodrugs of Cox-2 inhibitors is described in U.S. Pat. No. 5,932,598.

The cyclooxygenase-2 selective inhibitor of the present invention can be, for example, the Cox-2 selective inhibitor meloxicam, Formula B-1 (CAS registry number 71125-38-7), or-a pharmaceutically acceptable salt or prodrug thereof.

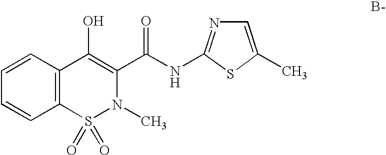

In another embodiment of the invention the cyclooxygenase-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

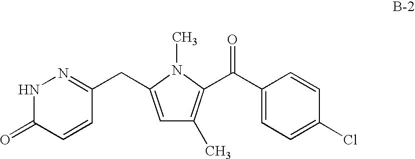

In a another embodiment of the invention the cyclooxygenase-2 selective inhibitor is of the chromene/chroman structural class that is a substituted benzopyran or a substituted benzopyran analog, and even more preferably selected from the group consisting of substituted benzothiopyrans, dihydroquinolines, or dihydronaphthalenes having the structure of any one of the compounds having a structure shown by general Formulas I, II, III, IV, V, and VI, shown below, and possessing, by way of example and not limitation, the structures disclosed in Table 1, including the diastereomers, enantiomers, racemates, tautomers, salts, esters, amides and prodrugs thereof.

Benzopyrans that can serve as a cyclooxygenase-2 selective inhibitor of the present invention include substituted benzopyran derivatives that are described in U.S. Pat. No. 6,271,253. One such class of compounds is defined by the general formula shown below in formulas I:

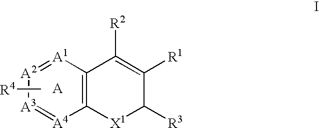

wherein X¹ is selected from O, S, CR^{c} R^{b} and NR^{a};

wherein R^{a} is selected from hydrido, C₁-C₃-alkyl, (optionally substituted phenyl)-C₁-C₃-alkyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b} R^{c} forms a 3-6 membered cycloalkyl ring;

wherein R¹ is selected from carboxyl, aminocarbonyl, C₁-C₆-alkylsulfonylaminocarbonyl and C₁-C₆-alkoxycarbonyl; wherein R² is selected from hydrido, phenyl, thienyl, C₁-C₆-alkyl and C₂-C₆-alkenyl;

wherein R³ is selected from C₁-C₃-perfluoroalkyl, chloro, C₁-C₆-alkylthio, C₁-C₆-alkoxy, nitro, cyano and cyano-C₁-C₃-alkyl;

wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and

wherein the A ring atoms A¹, A², A³ and A⁴ are independently selected from carbon and nitrogen with the proviso that at least two of A¹, A², A³ and A⁴ are carbon;

or wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

Another class of benzopyran derivatives that can serve as the Cox-2 selective inhibitor of the present invention includes a compound having the structure of formula II:

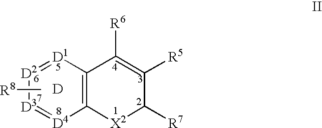

wherein X² is selected from O, S, CR^{c} R^{b} and NR^{a};

wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{c} R^{b} form a cyclopropyl ring;

wherein R⁵ is selected from carboxyl, aminocarbonyl, C₁-C₆-alkylsulfonylaminocarbonyl and C₁-C₆-alkoxycarbonyl;

wherein R⁶ is selected from hydrido, phenyl, thienyl, C₂-C₆-alkynyl and C₂-C₆-alkenyl;

wherein R⁷ is selected from C₁-C₃-perfluoroalkyl, chloro, C₁-C₆-alkylthio, C₁-C₆-alkoxy, nitro, cyano and cyano-C₁-C₃-alkyl; wherein R⁸ is one or more radicals independently selected from hydrido, halo, C_{1 -C}₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C_{1 -C}₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and

wherein the D ring atoms D¹, D², D³ and D⁴ are independently selected from carbon and nitrogen with the proviso that at least two of D¹, D², D³ and D⁴ are carbon; or

wherein R⁸ together with ring D forms a radical selected from naphthyl, quinolyl, isoquinolyl, quinolizinyl, quinoxalinyl and dibenzofuryl; or an isomer or pharmaceutically acceptable salt thereof.

Other benzopyran Cox-2 selective inhibitors useful in the practice of the present invention are described in U.S. Pat. Nos. 6,034,256 and 6,077,850. The general formula for these compounds is shown in formula III:

Formula III is:

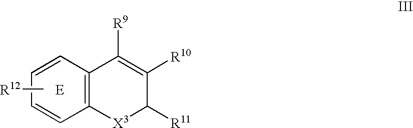

wherein X³ is selected from the group consisting of O or S or NR^{a};

wherein R^{a} is alkyl;

wherein R⁹ is selected from the group consisting of H and aryl;

wherein R¹⁰ is selected from the group consisting of carboxyl, aminocarbonyl, alkylsulfonylaminocarbonyl and alkoxycarbonyl; wherein R¹¹ is selected from the group consisting of haloalkyl, alkyl, aralkyl, cycloalkyl and aryl optionally substituted with one or more radicals selected from alkylthio, nitro and alkylsulfonyl; and

wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or

wherein R¹² together with ring E forms a naphthyl radical; or an isomer or pharmaceutically acceptable salt thereof; and including the diastereomers, enantiomers, racemates, tautomers, salts, esters, amides and prodrugs thereof.

A related class of compounds useful as cyclooxygenase-2 selective inhibitors in the present invention is described by Formulas IV and V:

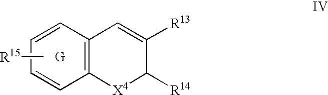

wherein X⁴ is selected from O or S or NR^{a};

wherein R^{a} is alkyl;

wherein R¹³ is selected from carboxyl, aminocarbonyl, alkylsulfonylaminocarbonyl and alkoxycarbonyl;

wherein R¹⁴ is selected from haloalkyl, alkyl, aralkyl, cycloalkyl and aryl optionally substituted with one or more radicals selected from alkylthio, nitro and alkylsulfonyl; and

wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;

or wherein R¹⁵ together with ring G forms a naphthyl radical; or an isomer or pharmaceutically acceptable salt thereof.

Formula V is:

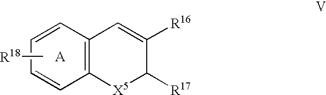

wherein:

X⁵ is selected from the group consisting of O or S or NR^{b};

R^{b} is alkyl;

R¹⁶ is selected from the group consisting of carboxyl, aminocarbonyl, alkylsulfonylaminocarbonyl and alkoxycarbonyl;

R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁸ together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of lower haloalkyl, lower cycloalkyl and phenyl; and

R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or

wherein R¹⁸ together with ring A forms a naphthyl radical; or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is carboxyl;

R¹⁷ is lower haloalkyl; and

R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R¹⁸ together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or

wherein R² together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting trifluoromethyl and pentafluoroethyl; and

R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N, N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹⁸ together with ring A forms a naphthyl radical;

or an isomer or prodrug thereof.

The cyclooxygenase-2 selective inhibitor of the present invention can also be a compound having the structure of Formula VI:

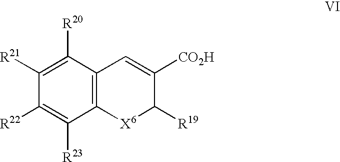

wherein:

X⁶ is selected from the group consisting of O and S;

R¹⁹ is lower haloalkyl;

R²⁰ is selected from the group consisting of hydrido, and halo;

R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;

R²² is selected from the group consisting of hydrido, lower alkyl, halo, lower alkoxy, and aryl; and

R²³ is selected from the group consisting of the group consisting of hydrido, halo, lower alkyl, lower alkoxy, and aryl;

or an isomer or prodrug thereof.

The cyclooxygenase-2 selective inhibitor can also be a compound of having the structure of Formula VI, wherein:

X⁶ is selected from the group consisting of O and S;

R¹⁹ is selected from the group consisting of trifluoromethyl and pentafluoroethyl;

R²⁰ is selected from the group consisting of hydrido, chloro, and fluoro;

R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R²² is selected from the group consisting of hydrido, methyl, ethyl, isopropyl, tert-butyl, chloro, methoxy, diethylamino, and phenyl; and

R²³ is selected from the group consisting of hydrido, chloro, bromo, fluoro, methyl, ethyl, tert-butyl, methoxy, and phenyl;

or an isomer or prodrug thereof.

TABLE 1
Examples of Chromene Cox-2 Selective Inhibitors

| Compound | |
|---|---|
| Number | Structural Formula |
| B-3 | 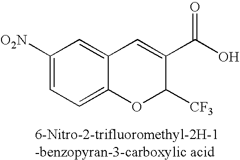 |
| B-4 | 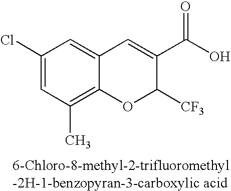 |
| B-5 | 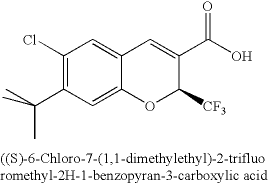 |
| B-6 | 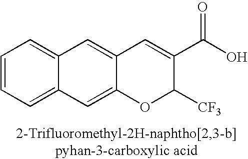 |
| B-7 | 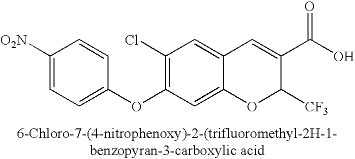 |
| B-8 | 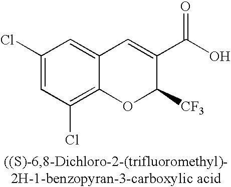 |
| B-9 | 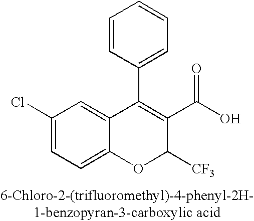 |
| B-10 | 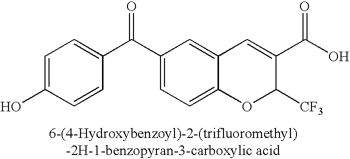 |
| B-11 | 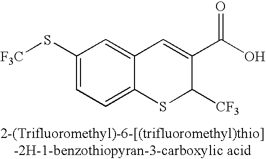 |
| B-12 | 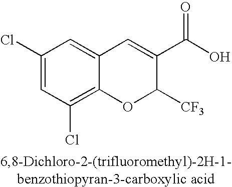 |
| B-13 | 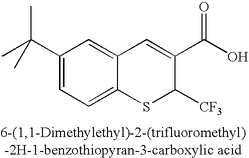 |
| B-14 | 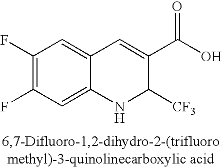 |
| B-15 | 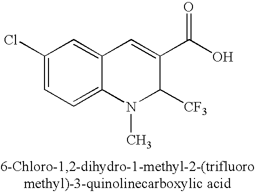 |
| B-16 | 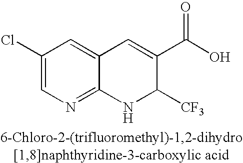 |
| B-17 | 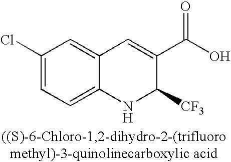 |

Examples of specific compounds that are useful for the cycloxygenase-2 selective inhibitor include (without limitation):

a1) 8-acetyl-3-(4-flourophenyl)-2-(4-methylsulfonyl)phenyl-imidazo(1,2-a)pyridine;

a2) 5,5-dimethyl-4-(4-methylsulfonyl)phenyl-3-phenyl-2-(5H)-furanone;

a3) 5-(4-fluorophenyl)-1-[4-(methylsulfonyl)phenyl]-3-(trifluoromethyl)pyrazole;

a4) 4-(4-fluorophenyl)-5-[4-(methylsulfonyl)phenyl]-1-phenyl-3-(trifluoromethyl)pyrazole;

a5) 4-(5-(4-chlorophenyl)-3-(4-methoxyphenyl)-1H-pyrazol-1-yl)benzenesulfonamide

a6) 4-(3,5-bis(4-methylphenyl)-1H-pyrazol-1-yl)benzenesulfonamide;

a7) 4-(5-(4-chlorophenyl)-3-phenyl-1H-pyrazol-1-yl)benzenesulfonamide;

a8) 4-(3,5-bis(4-methoxyphenyl)-1H-pyrazol-1-yl)benzenesulfonamide;

a9) 4-(5-(4-chlorophenyl)-3-(4-methylphenyl)-1H-pyrazol-1-yl)benzenesulfonamide;

a10) 4-(5-(4-chlorophenyl)-3-(4-nitrophenyl)-1H-pyrazol-1-yl)benzenesulfonamide;

b1) 4-(5-(4-chlorophenyl)-3-(5-chloro-2-thienyl)-1H-pyrazol-1-yl)benzenesulfonamide;

b2) 4-(4-chloro-3,5-diphenyl-1H-pyrazol-1-yl)benzenesulfonamide

b3) 4-[5-(4-chlorophenyl)-3-(trifluoromethyl)-1H-pyrazol-1-yl]benzenesulfonamide;

b4) 4-[5-phenyl-3-(trifluoromethyl)-1H-pyrazol-1-yl]benzenesulfonamide;

b5) 4-[5-(4-fluorophenyl)-3-(trifluoromethyl)-1H-pyrazol-1-yl]benzenesulfonamide;

b6) 4-[5-(4-methoxyphenyl)-3-(trifluoromethyl)-1H-pyrazol-1-yl]benzenesulfonamide;

b7) 4-[5-(4-chlorophenyl)-3-(difluoromethyl)- 1H-pyrazol-1-yl]benzenesulfonamide;

b8) 4-[5-(4-methylphenyl)-3-(trifluoromethyl)-1H-pyrazol-1-yl]benzenesulfonamide;

b9) 4-[4-chloro-5-(4-chlorophenyl)-3-(trifluoromethyl)-1H-pyrazol-1-yl]benzenesulfonamide;

b 10) 4-[3-(difluoromethyl)-5-(4-methylphenyl)-1H-pyrazol-1-yl]benzenesulfonamide;

c1) 4-[3-(difluoromethyl)-5-phenyl-1H-pyrazol-1-yl]benzenesulfonamide;

c2) 4-[3-(difluoromethyl)-5-(4-methoxyphenyl)-1H-pyrazol-1-yl]benzenesulfonamide;

c3) 4-[3-cyano-5-(4-fluorophenyl)-1H-pyrazol-1-yl]benzenesulfonamide;

c4) 4-[3-(difluoromethyl)-5-(3-fluoro-4-methoxyphenyl)-1H-pyrazol-1-yl]benzenesulfonamide;

c5) 4-[5-(3-fluoro-4-methoxyphenyl)-3-(trifluoromethyl)-1H-pyrazol-1-yl]benzenesulfonamide;

c6) 4-[4-chloro-5-phenyl-1H-pyrazol-1-yl]benzenesulfonamide;

c7) 4-[5-(4-chlorophenyl)-3-(hydroxymethyl)-1H-pyrazol-1-yl]benzenesulfonamide;

c8) 4-[5-(4-(N,N-dimethylamino)phenyl)-3-(trifluoromethyl)-1H-pyrazol-1-yl]benzenesulfonamide;

c9) 5-(4-fluorophenyl)-6-[4-(methylsulfonyl)phenyl]spiro[2.4]hept-5-ene;

c10) 4-[6-(4-fluorophenyl)spiro[2.4]hept-5-en-5-yl]benzenesulfonamide;

d1) 6-(4-fluorophenyl)-7-[4-(methylsulfonyl)phenyl]spiro[3.4]oct-6-ene;

d2) 5-(3-chloro-4-methoxyphenyl)-6-[4-(methylsulfonyl)phenyl]spiro[2 .4]hept-5-ene;

d3) 4-[6-(3-chloro-4-methoxyphenyl)spiro[2.4]hept-5-en-5-yl]benzenesulfonamide;

d4) 5-(3,5-dichloro-4-methoxyphenyl)-6-[4-(methylsulfonyl)phenyl]spiro[2.4]hept-5-ene;

d5) 5-(3-chloro-4-fluorophenyl)-6-[4-(methylsulfonyl)phenyl]spiro[2.4]hept-5-ene;

d6) 4-[6-(3,4-dichlorophenyl)spiro[2.4]hept-5-en-5-yl]benzenesulfonamide;

d7) 2-(3-chloro-4-fluorophenyl)-4-(4-fluorophenyl)-5-(4-methylsulfonylphenyl)thiazole;

d8) 2-(2-chlorophenyl)-4-(4-fluorophenyl)-5-(4-methylsulfonylphenyl)thiazole;

d9) 5-(4-fluorophenyl)-4-(4-methylsulfonylphenyl)-2-methylthiazole;

d10) 4-(4-fluorophenyl)-5-(4-methylsulfonylphenyl)-2-trifluoromethylthiazole;

e1) 4-(4-fluorophenyl)-5-(4-methylsulfonylphenyl)-2-(2-thienyl)thiazole;

e2) 4-(4-fluorophenyl)-5-(4-methylsulfonylphenyl)-2-benzylaminothiazole;

e3) 4-(4-fluorophenyl)-5-(4-methylsulfonylphenyl)-2-( 1-propylamino)thiazole;

e4) 2-[(3,5-dichlorophenoxy)methyl)-4-(4-fluorophenyl)-5-[4-(methylsulfonyl)phenyl]thiazole;

e5) 5-(4-fluorophenyl)-4-(4-methylsulfonylphenyl)-2-trifluoromethylthiazole;

e6) 1-methylsulfonyl-4-[1,1-dimethyl-4-(4-fluorophenyl)cyclopenta-2,4-dien-3-yl]benzene;

e7) 4-[4-(4-fluorophenyl)-1,1-dimethylcyclopenta-2,4-dien-3-yl]benzenesulfonamide;

e8) 5-(4-fluorophenyl)-6-[4-(methylsulfonyl)phenyl]spiro[2.4]hepta-4,6-diene;

e9) 4-[6-(4-fluorophenyl)spiro[2.4]hepta-4,6-dien-5-yl]benzenesulfonamide;

e10) 6-(4-fluorophenyl)-2-methoxy-5-[4-(methylsulfonyl)phenyl]-pyridine-3-carbonitrile;

f1) 2-bromo-6-(4-fluorophenyl)-5-[4-(methylsulfonyl)phenyl]-pyridine-3-carbonitrile;

f2) 6-(4-fluorophenyl)-5-[4-(methylsulfonyl)phenyl]-2-phenyl-pyridine-3-carbonitrile;

f3) 4-[2-(4-methylpyridin-2-yl)-4-(trifluoromethyl)-1H-imidazol-1-yl]benzenesulfonamide;

f4) 4-[2-(5-methylpyridin-3-yl)-4-(trifluoromethyl)-1H-imidazol-1-yl]benzenesulfonamide;

f5) 4-[2-(2-methylpyridin-3-yl)-4-(trifluoromethyl)- 1H-imidazol- 1-yl]benzenesulfonamide;

f6) 3-[1-[4-(methylsulfonyl)phenyl]-4-(trifluoromethyl)-1H-imidazol-2-yl]pyridine;

f7) 2-[1-[4-(methylsulfonyl)phenyl-4-(trifluoromethyl)-1H-imidazol-2-yl]pyridine;

f8) 2-methyl-4-[1-[4-(methylsulfonyl)phenyl-4-(trifluoromethyl)-1H-imidazol-2-yl]pyridine;

f9) 2-methyl-6-[1-[4-(methylsulfonyl)phenyl-4-(trifluoromethyl)-1H-imidazol-2-yl]pyridine;

f10) 4-[2-(6-methylpyridin-3-yl)-4-(trifluoromethyl)-1H-imidazol-1-yl]benzenesulfonamide;

g1) 2-(3,4-difluorophenyl)-1-[4-(methylsulfonyl)phenyl]-4-(trifluoromethyl)-1H-imidazole;

g2) 4-[2-(4-methylphenyl)-4-(trifluoromethyl)-1H-imidazol-1-yl]benzenesulfonamide;

g3) 2-(4-chlorophenyl)-1-[4-(methylsulfonyl)phenyl]-4-methyl-1H-imidazole;

g4) 2-(4-chlorophenyl)-1-[4-(methylsulfonyl)phenyl]-4-phenyl-1H-imidazole;

g5) 2-(4-chlorophenyl)-4-(4-fluorophenyl)-1-[4-(methylsulfonyl)phenyl]-1H-imidazole;

g6) 2-(3-fluoro-4-methoxyphenyl)-1-[4-(methylsulfonyl)phenyl-4-(trifluoromethyl)-1H-imidazole;

g7) 1-[4-(methylsulfonyl)phenyl]-2-phenyl-4-trifluoromethyl-1H-imidazole;

g8) 2-(4-methylphenyl)-1-[4-(methylsulfonyl)phenyl]-4-trifluoromethyl-1H-imidazole;

g9) 4-[2-(3-chloro-4-methylphenyl)-4-(trifluoromethyl)-1H-imidazol-1-yl]benzenesulfonamide;

g10) 2-(3-fluoro-5-methylphenyl)-1-[4-(methylsulfonyl)phenyl]-4-(trifluoromethyl)-1H-imidazole;

h1) 4-[2-(3-fluoro-5-methylphenyl)-4-(trifluoromethyl)-1H-imidazol-1-yl]benzenesulfonamide;

h2) 2-(3-methylphenyl)-1-[4-(methylsulfonyl)phenyl]-4-trifluoromethyl-1H-imidazole;

h3) 4-[2-(3-methylphenyl)-4-trifluoromethyl-1H-imidazol-1-yl]benzenesulfonamide;

h4) 1-[4-(methylsulfonyl)phenyl]-2-(3-chlorophenyl)-4-trifluoromethyl-1H-imidazole;

h5) 4-[2-(3-chlorophenyl)-4-trifluoromethyl-1H-imidazol-1-yl]benzenesulfonamide;

h6) 4-[2-phenyl-4-trifluoromethyl-1H-imidazol- 1-yl]benzenesulfonamide;

h7) 4-[2-(4-methoxy-3-chlorophenyl)-4-trifluoromethyl-1H-imidazol-1-yl]benzenesulfonamide;

h8) 1-allyl-4-(4-fluorophenyl)-3-[4-(methylsulfonyl)phenyl]-5-(trifluoromethyl)-1H-pyrazole;

h10) 4-[1-ethyl-4-(4-fluorophenyl)-5-(trifluoromethyl)-1H-pyrazol-3-yl]benzenesulfonamide;

i1) N-phenyl-[4-(4-luorophenyl)-3-[4-(methylsulfonyl)phenyl]-5-(trifluoromethyl)-1H-pyrazol-1-yl]acetamide;

i2) ethyl [4-(4-fluorophenyl)-3-[4-(methylsulfonyl)phenyl]-5-(trifluoromethyl)-1H-pyrazol-1-yl]acetate;

i3) 4-(4-fluorophenyl)-3-[4-(methylsulfonyl)phenyl]-1-(2-phenylethyl)-1H-pyrazole;

i4) 4-(4-fluorophenyl)-3-[4-(methylsulfonyl)phenyl]-1-(2-phenylethyl)-5-(trifluoromethyl)pyrazole;

i5) 1-ethyl-4-(4-fluorophenyl)-3-[4-(methylsulfonyl)phenyl]-5-(trifluoromethyl)-1H-pyrazole;

i6) 5-(4-fluorophenyl)-4-(4-methylsulfonylphenyl)-2-trifluoromethyl-1H-imidazole;

i7) 4-[4-(methylsulfonyl)phenyl]-5-(2-thiophenyl)-2-(trifluoromethyl)-1H-imidazole;

i8) 5-(4-fluorophenyl)-2-methoxy-4-[4-(methylsulfonyl)phenyl]-6-(trifluoromethyl)pyridine;

i9) 2-ethoxy-5-(4-fluorophenyl)-4-[4-(methylsulfonyl)phenyl]-6-(trifluoromethyl)pyridine;

i10) 5-(4-fluorophenyl)-4-[4-(methylsulfonyl)phenyl]-2-(2-propynyloxy)-6-(trifluoromethyl)pyridine;

j1) 2-bromo-5-(4-fluorophenyl)-4-[4-(methylsulfonyl)phenyl]-6-(trifluoromethyl)pyridine;

j2) 4-[2-(3-chloro-4-methoxyphenyl)-4,5-difluorophenyl]benzenesulfonamide;

j3) 1-(4-fluorophenyl)-2-[4-(methylsulfonyl)phenyl]benzene;

j4) 5-difluoromethyl-4-(4-methylsulfonylphenyl)-3-phenylisoxazole;

j5) 4-[3-ethyl-5-phenylisoxazol-4-yl]benzenesulfonamide;

j6) 4-[5-difluoromethyl-3-phenylisoxazol-4-yl]benzenesulfonamide;

j7) 4-[5-hydroxymethyl-3-phenylisoxazol-4-yl]benzenesulfonamide;

j8) 4-[5-methyl-3-phenyl-isoxazol-4-yl]benzenesulfonamide;

j9) 1-[2-(4-fluorophenyl)cyclopenten-1-yl]-4-(methylsulfonyl)benzene;

j10) 1-[2-(4-fluoro-2-methylphenyl)cyclopenten-1-yl]-4-(methylsulfonyl)benzene;

k1) 1-[2-(4-chlorophenyl)cyclopenten-1-yl]-4-(methylsulfonyl)benzene;

k2) 1-[2-(2,4-dichlorophenyl)cyclopenten-1-yl]-4-(methylsulfonyl)benzene;

k3) 1-[2-(4-trifluoromethylphenyl)cyclopenten-1-yl]-4-(methylsulfonyl)benzene;

k4) 1-[2-(4-methylthiophenyl)cyclopenten-1-yl]-4-(methylsulfonyl)benzene;

k5) 1-[2-(4-fluorophenyl)-4,4-dimethylcyclopenten-1-yl]-4-(methylsulfonyl)benzene;

k6) 4-[2-(4-fluorophenyl)-4,4-dimethylcyclopenten-1-yl]benzenesulfonamide;

k7) 1-[2-(4-chlorophenyl)-4,4-dimethylcyclopenten-1-yl]-4-(methylsulfonyl)benzene;

k8) 4-[2-(4-chlorophenyl)-4,4-dimethylcyclopenten-1-yl]benzenesulfonamide;

k9) 4-[2-(4-fluorophenyl)cyclopenten-1-yl]benzenesulfonamide;

k10) 4-[2-(4-chlorophenyl)cyclopenten-1-yl]benzenesulfonamide;

l1) 1-[2-(4-methoxyphenyl)cyclopenten-1-yl]-4-(methylsulfonyl)benzene;

l2) 1-[2-(2,3-difluorophenyl)cyclopenten-1-yl]-4-(methylsulfonyl)benzene;

l3) 4-[2-(3-fluoro-4-methoxyphenyl)cyclopenten-1-yl]benzenesulfonamide;

l4) 1-[2-(3-chloro-4-methoxyphenyl)cyclopenten-1-yl]-4-(methylsulfonyl)benzene;

l5) 4-[2-(3-chloro-4-fluorophenyl)cyclopenten-1-yl]benzenesulfonamide;

l6) 4-[2-(2-methylpyridin-5-yl)cyclopenten-1-yl]benzenesulfonamide;

l7) ethyl 2-[4-(4-fluorophenyl)-5-[4-(methylsulfonyl) phenyl]oxazol-2-yl]-2-benzyl-acetate;

l8) 2-[4-(4-fluorophenyl)-5-[4-(methylsulfonyl)phenyl]oxazol-2-yl]acetic acid; l9) 2-(tert-butyl)-4-(4-fluorophenyl)-5-[4-(methylsulfonyl)phenyl]oxazole; l10) 4-(4-fluorophenyl)-5-[4-(methylsulfonyl)phenyl]-2-phenyloxazole;

m1) 4-(4-fluorophenyl)-2-methyl-5-[4-(methylsulfonyl)phenyl]oxazole; and

m2) 4-[5-(3-fluoro-4-methoxyphenyl)-2-trifluoromethyl-4-oxazolyl]benzenesulfonamide.

m3) 6-chloro-2-trifluoromethyl-2H-1-benzopyran-3-carboxylic acid;

m4) 6-chloro-7-methyl-2-trifluoromethyl-2H-1-benzopyran-3-carboxylic acid;

m5) 8-(1-methylethyl)-2-trifluoromethyl-2H-1-benzopyran-3-carboxylic acid;

m6) 6-chloro-7-(1,1-dimethylethyl)-2-trifluoromethyl-2H-1-benzopyran-3-carboxylic acid;

m7) 6-chloro-8-( 1-methylethyl)-2-trifluoromethyl-2H-1-benzopyran-3-carboxylic acid;

m8) 2-trifluoromethyl-3H-naphthopyran-3-carboxylic acid;

m9) 7-(1,1-dimethylethyl)-2-trifluoromethyl-2H-1-benzopyran-3-carboxylic acid;

m10) 6-bromo-2-trifluoromethyl-2H-1-benzopyran-3-carboxylic acid;

n1) 8-chloro-2-trifluoromethyl-2H-1-benzopyran-3-carboxylic acid;

n2) 6-trifluoromethoxy-2-trifluoromethyl-2H-1-benzopyran-3-carboxyiic acid;

n3) 5,7-dichloro-2-trifluoromethyl-2H-1-benzopyran-3-carboxylic acid;

n4) 8-phenyl-2-trifluoromethyl-2H-1-benzopyran-3-carboxylic acid;

n5) 7,8-dimethyl-2-trifluoromethyl-2H-1-benzopyran-3-carboxylic acid;

n6) 6,8-bis(dimethylethyl)-2-trifluoromethyl-2H-1-benzopyran-3-carboxylic acid;

n7) 7-(1-methylethyl)-2-trifluoromethyl-2H-1-benzopyran-3-carboxylic acid;

n8) 7-phenyl-2-trifluoromethyl-2H-1-benzopyran-3-carboxylic acid;

n9) 6-chloro-7-ethyl-2-trifluoromethyl-2H-1-benzopyran-3-carboxylic acid;

n10) 6-chloro-8-ethyl-2-trifluoromethyl-2H-1-benzopyran-3-carboxylic acid;

o1) 6-chloro-7-phenyl-2-trifluoromethyl-2H-1-benzopyran-3-carboxylic acid;

o2) 6,7-dichloro-2-trifluoromethyl-2H-1-benzopyran-3-carboxylic acid;

o3) 6,8-dichloro-2-trifluoromethyl-2H-1-benzopyran-3-carboxylic acid;

o4) 2-trifluoromethyl-3H-naptho[2,1-b]pyran-3-carboxylic acid;

o5) 6-chloro-8-methyl-2-trifluoromethyl-2H-1-benzopyran-3-carboxylic acid;

o6) 8-chloro-6-methyl-2-trifluoromethyl-2H-1-benzopyran-3-carboxylic acid;

o7) 8-chloro-6-methoxy-2-trifluoromethyl-2H-1-benzopyran-3-carboxylic acid;

o8) 6-bromo-8-chloro-2-trifluoromethyl-2H-1-benzopyran-3-carboxylic acid;

o9) 8-bromo-6-fluoro-2-trifluoromethyl-2H-1-benzopyran-3-carboxylic acid;

o10) 8-bromo-6-methyl-2-trifluoromethyl-2H-1-benzopyran-3-carboxylic acid;

p1) 8-bromo-5-fluoro-2-trifluoromethyl-2H-1-benzopyran-3-carboxylic acid;

p2) 6-chloro-8-fluoro-2-trifluoromethyl-2H-1-benzopyran-3-carboxylic acid;

p3) 6-bromo-8-methoxy-2-trifluoromethyl-2H-1-benzopyran-3-carboxylic acid;

p4) 6-[[(phenylmethyl)amino]sulfonyl]-2-trifluoromethyl-2H-1-benzopyran-3-carboxylic acid;

p5) 6-[(dimethylamino)sulfonyl]-2-trifluoromethyl-2H-1-benzopyran-3-carboxylic acid;

p6) 6-[(methylamino)sulfonyl]-2-trifluoromethyl-2H-1-benzopyran-3-carboxylic acid;

p7) 6-[(4-morpholino)sulfonyl]-2-trifluoromethyl-2H-1-benzopyran-3-carboxylic acid;

p8) 6-[(1,1-dimethylethyl)aminosulfonyl]-2-trifluoromethyl-2H-1-benzopyran-3-carboxylic acid;

p9) 6-[(2-methylpropyl)aminosulfonyl]-2-trifluoromethyl-2H-1-benzopyran-3-carboxylic acid;

p10) 6-methylsulfonyl-2-trifluoromethyl-2H-1-benzopyran-3-carboxylic acid;

q1) 8-chloro-6-[[(phenylmethyl)amino]sulfonyl]-2-trifluoromethyl-2H-1-benzopyran-3-carboxylic acid;

q2) 6-phenylacetyl-2-trifluoromethyl-2H-1-benzopyran-3-carboxylic acid;

q3) 6,8-dibromo-2-trifluoromethyl-2H-1-benzopyran-3-carboxylic acid;

q4) 8-chloro-5,6-dimethyl-2-trifluoromethyl-2H-1-benzopyran-3-carboxylic acid;

q5) 6,8-dichloro-(S)-2-trifluoromethyl-2H-1-benzopyran-3-carboxylic acid;

q6) 6-benzylsulfonyl-2-trifluoromethyl-2H-1-benzopyran-3-carboxylic acid;

q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;

q 10) 7-(1,1-dimethylethyl)-2-pentafluoroethyl-2H-1-benzopyran-3-carboxylic acid;

r1) 5,5-dimethyl-3-(3-fluorophenyl)-4-(4-methyl-sulphonyl-2(5H)-fluranone;

r2) 6-chloro-2-trifluoromethyl-2H-1-benzothiopyran-3-carboxylic acid;

r3) 4-[5-(4-chlorophenyl)-3-(trifluoromethyl)-1H-pyrazol-1-yl]benzenesulfonamide;

r4) 4-[5-(4-methylphenyl)-3-(trifluoromethyl)-1H-pyrazol-1-yl]benzenesulfonamide;

r5) 4-[5-(3-fluoro-4-methoxyphenyl)-3-(difluoromethyl)-1H-pyrazol-1-yl]benzenesulfonamide;

r6) 3-[1-[4-(methylsulfonyl)phenyl]-4-trifluoromethyl-1H-imidazol-2-yl]pyridine;

r7) 2-methyl-5-[1-[4-(methylsulfonyl)phenyl]-4-trifluoromethyl-1H-imidazol-2-yl]pyridine;

r8) 4-[2-(5-methylpyridin-3-yl)-4-(trifluoromethyl)-1H-imidazol-1-yl]benzenesulfonamide;

r9) 4-[5-methyl-3-phenylisoxazol-4-yl]benzenesulfonamide;

r10) 4-[5-hydroxymethyl-3-phenylisoxazol-4-yl]benzenesulfonamide;

s1) [2-trifluoromethyl-5-(3,4-difluorophenyl)-4-oxazolyl]benzenesulfonamide;

s2) 4-[2-methyl-4-phenyl-5-oxazolyl]benzenesulfonamide; or

s3) 4-[5-(3-fluoro-4-methoxyphenyl-2-trifluoromethyl)-4-oxazolyl]benzenesulfonamide;

or a pharmaceutically acceptable salt or prodrug thereof.

In a further preferred embodiment of the invention the cyclooxygenase inhibitor can be selected from the class of tricyclic cyclooxygenase-2 selective inhibitors represented by the general structure of formula VII:

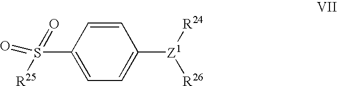

wherein:

Z¹ is selected from the group consisting of partially unsaturated or unsaturated heterocyclyl and partially unsaturated or unsaturated carbocyclic rings;

R is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R²⁵ is selected from the group consisting of methyl or amino; and

R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N- arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-arylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the cyclooxygenase-2 selective inhibitor represented by the above Formula VII is selected from the group of compounds, illustrated in Table 2, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 2
Examples of Tricyclic COX-2 Selective Inhibitors

| Compound | |
|---|---|
| Number | Structural Formula |
| B-18 | 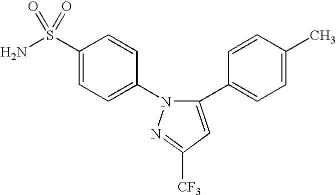 |
| B-19 | 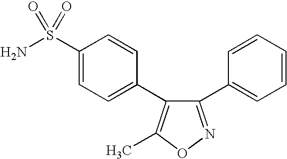 |
| B-20 | 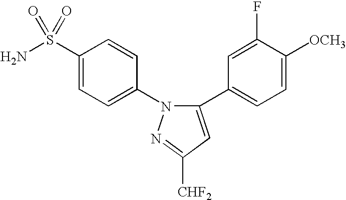 |
| B-21 | 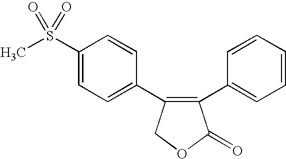 |
| B-22 | 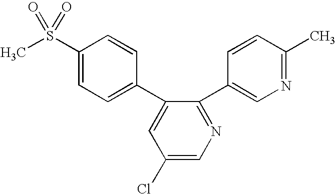 |
| B-23 | 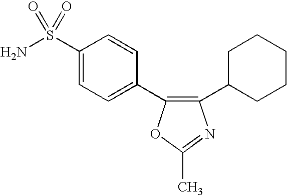 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-24, which is a therapeutically effective prodrug of the tricyclic cyclooxygenase-2 selective inhibitor valdecoxib, B-19, (See, e.g., U.S. Pat. No. 5,633,272), may be advantageously employed as a source of a cyclooxygenase inhibitor.

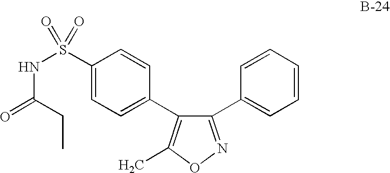

A preferred form of parecoxib is sodium parecoxib.

In another embodiment of the invention, the compound ABT-963 having the formula B-25 that has been previously described in International Publication number WO 00/24719, is another tricyclic cyclooxygenase-2 selective inhibitor which may be advantageously employed.

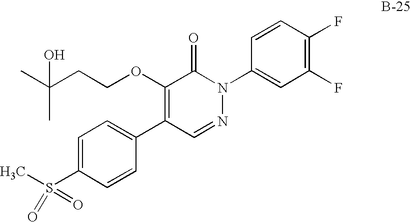

In a further embodiment of the invention, the cyclooxygenase inhibitor can be selected from the class of phenylacetic acid derivative cyclooxygenase-2 selective inhibitors represented by the general structure of Formula VII:

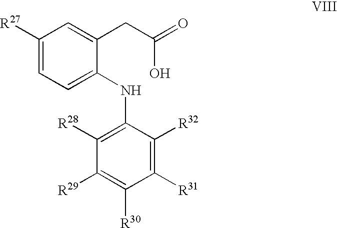

wherein:

R²⁷ is methyl, ethyl, or propyl;

R²⁸ is chloro or fluoro;

R²⁹ is hydrogen, fluoro, or methyl;

R³⁰ is hydrogen, fluoro, chloro, methyl, ethyl, methoxy, ethoxy or hydroxy;

R³¹ is hydrogen, fluoro, or methyl; and

R³² is chloro, fluoro, trifluoromethyl, methyl, or ethyl, provided that R²⁸, R²⁹, R³⁰ and R³¹ are not all fluoro when R²⁷ is ethyl and R³⁰ is H.

A phenylacetic acid derivative cyclooxygenase-2 selective inhibitor that is described in WO 99/11605 is a compound that has the structure shown in Formula VIII,

wherein:

R²⁷ is ethyl;

R²⁸ and R³⁰ are chloro;

R²⁹ and R³¹ are hydrogen; and

R³² is methyl.

Another phenylacetic acid derivative cyclooxygenase-2 selective inhibitor is a compound that has the structure shown in Formula VIII,

wherein:

R²⁷ is propyl;

R²⁸ and R³⁰ are chloro;

R²⁹ and R³¹ are methyl; and

R³² is ethyl.

Another phenylacetic acid derivative cyclooxygenase-2 selective inhibitor that is described in WO 02/20090 is a compound that is referred to as COX-189 (also termed lumiracoxib), having CAS Reg. No. 220991-20-8, and having the structure shown in Formula VIII,

wherein:

R²⁷ is methyl;

R²⁸ is fluoro;

R³² is chloro; and

R²⁹, R³⁰, and R³¹ are hydrogen.

Compounds that have a structure similar to that shown in Formula VIII, which can serve as the Cox-2 selective inhibitor of the present invention, are described in U.S. Pat. Nos. 6,310,099, 6,291,523, and 5,958,978.

Other cyclooxygenase-2 selective inhibitors that can be used in the present invention have the general structure shown in formula IX, where the J group is a carbocycle or a heterocycle. Preferred embodiments have the structure:

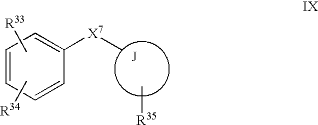

wherein:

X is O; J is 1-phenyl; R³³ is 2-NHSO₂CH₃; R³⁴ is 4-NO₂; and there is no R³⁵ group, (nimesulide), and

X is O; J is 1-oxo-inden-5-yl; R³³ is 2-F; R³⁴ is 4-F; and R³⁵ is 6- NHSO₂CH₃, (flosulide); and

X is O; J is cyclohexyl; R³³ is 2-NHSO₂CH₃; R³⁴ is 5-NO₂; and there is no R³⁵ group, (NS-398); and

X is S; J is 1-oxo-inden-5-yl; R³³ is 2-F; R³⁴ is 4-F; and R³⁵ is 6-N⁻SO₂CH₃.Na⁺, (L-745337); and

X is S; J is thiophen-2-yl; R³³ is 4-F; there is no R³⁴ group; and R³⁵ is 5-NHSO₂CH₃, (RWJ-63556); and

X is O; J is 2-oxo-5(R)-methyl-5-(2,2,2-trifluoroethyl)furan-(5H)-3-yl; R³³ is 3-F; R³⁴ is 4-F; and R³⁵ is 4-(p-SO₂CH₃)C₆H₄, (L-784512).

Further information on the applications of the Cox-2 selective inhibitor N-(2-cyclohexyloxynitrophenyl) methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J.-P. et al., in Science Spectra, available at: http://www.gbhap.com/Science_Spectra/20-1-article.htm (06/06/2001); and Iwata, K. et al., in Jpn. J. Pharmacol., 75(2):191-194 (1997).

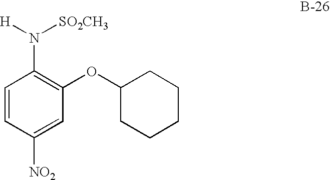

An evaluation of the anti-inflammatory activity of the cyclooxygenase-2 selective inhibitor, RWJ 63556, in a canine model of inflammation, was described by Kirchner et al., in J Pharmacol Exp Ther 282, 1094-1101 (1997).

Materials that can serve as the cyclooxygenase-2 selective inhibitor of the present invention include diarylmethylidenefuran derivatives that are described in U.S. Pat. No. 6,180,651. Such diarylmethylidenefuran derivatives have the general formula shown below in formula X:

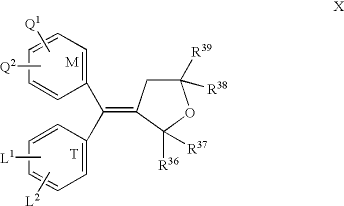

wherein:

the rings T and M independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms;

at least one of the substituents Q¹, Q², L¹ or L² is: an —S(O)ₙ—R group, in which n is an integer equal to 0, 1 or 2 and R is:

a lower alkyl radical having 1 to 6 carbon atoms or

a lower haloalkyl radical having 1 to 6 carbon atoms, or

an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q¹ and Q² or L¹ and L² are a methylenedioxy group; and

R³⁶, R³⁷, R³⁸ and R³⁹ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R³⁶, R³⁷ or R³⁸, R³⁹ are an oxygen atom, or

R³⁶, R³⁷ or R³⁸, R³⁹, together with the carbon atom to which they are attached, form a saturated hydrocarbon ring having from 3 to 7 carbon atoms;

or an isomer or prodrug thereof.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs. com/NEWS/Inflam1.htm, 10/04/2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀=0.39 mg/kg.

Compounds that may act as cyclooxygenase-2 selective inhibitors include multibinding compounds containing from 2 to 10 ligands covanlently attached to one or more linkers, as described in U.S. Pat. No. 6,395,724.

Compounds that may act as cyclooxygenase-2 inhibitors include conjugated linoleic acid that is described in U.S. Pat. No. 6,077,868.

Materials that can serve as a cyclooxygenase-2 selective inhibitor of the present invention include heterocyclic aromatic oxazole compounds that are described in U.S. Pat. Nos. 5,994,381 and 6,362,209. Such heterocyclic aromatic oxazole compounds have the formula shown below in formula XI:

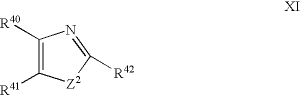

wherein:

Z² is an oxygen atom;

one of R⁴⁰ and R⁴¹ is a group of the formula

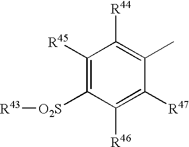

wherein:

R⁴³ is lower alkyl, amino or lower alkylamino; and

R⁴⁴, R⁴⁵, R⁴⁶ and R⁴⁷ are the same or different and each is hydrogen atom, halogen atom, lower alkyl, lower alkoxy, trifluoromethyl, hydroxy or amino, provided that at least one of R⁴⁴, R⁴⁵, R⁴⁶ and R⁴⁷ is not hydrogen atom, and the other is an optionally substituted cycloalkyl, an optionally substituted heterocyclic group or an optionally substituted aryl; and

R³⁰ is a lower alkyl or a halogenated lower alkyl, and a pharmaceutically acceptable salt thereof.

Cox-2 selective inhibitors that are useful in the subject method and compositions can include compounds that are described in U.S. Pat. Nos. 6,080,876 and 6,133,292, and described by formula XII:

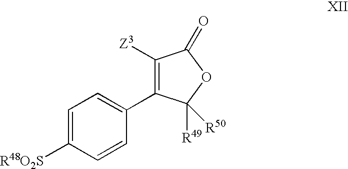

wherein:

Z³ is selected from the group consisting of:

(a) linear or branched C₁₋₆ alkyl,

(b) linear or branched C₁₋₆ alkoxy,

(c) unsubstituted, mono-, di- or tri-substituted phenyl or naphthyl wherein the substituents are selected from the group consisting of:

(1) hydrogen,

(2) halo,

(3) C₁₋₃ alkoxy,

(4) CN,

(5) C₁₋₃ fluoroalkyl

(6) C₁₋₃ alkyl,

(7) —CO₂H;

R⁴⁸ is selected from the group consisting of NH₂ and CH₃,

R⁴⁹ is selected from the group consisting of:

C₁₋₆ alkyl unsubstituted or substituted with C₃₋₆ cycloalkyl, and C₃₋₆ cycloalkyl;

R⁵⁰ is selected from the group consisting of:

C₁₋₆ alkyl unsubstituted or substituted with one, two or three fluoro atoms; and

C₃₋₆ cycloalkyl;

with the proviso that R⁴⁹ and R⁵⁰ are not the same.

Materials that can serve as cyclooxygenase-2 selective inhibitors include pyridines that are described in U.S. Pat. Nos. 6,369,275, 6,127,545, 6,130,334, 6,204,387, 6,071,936, 6,001,843 and 6,040,450, and which have the general formula described by formula XIII:

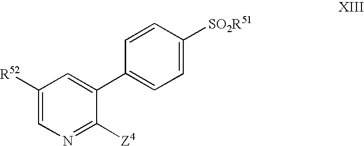

wherein:

R⁵¹ is selected from the group consisting of:

(a) CH₃,

(b) NH₂,

(c) NHC(O)CF₃,

(d) NHCH₃;

Z⁴ is a mono-, di-, or trisubstituted phenyl or pyridinyl (or the N-oxide thereof),

wherein the substituents are chosen from the group consisting of:

(a) hydrogen,

(b) halo,

(c) C₁₋₆ alkoxy,

(d) C₁₋₆ alkylthio,

(e) CN,

(f) C₁₋₆ alkyl,

(g) C₁₋₆ fluoroalkyl,

(h) N₃,

(i) —CO₂R⁵³,

(j) hydroxy,

(k) —C(R⁵⁴)(R⁵⁵)—OH,

(l) —C₁₋₆alkyl-CO₂—R⁵⁶,

(m) C₁₋₆fluoroalkoxy;

R⁵² is chosen from the group consisting of:

(a) halo,

(b) C₁₋₆alkoxy,

(c) C₁₋₆ alkylthio,

(d) CN,

(e) C₁₋₆ alkyl,

(f) C₁₋₆ fluoroalkyl,

(g) N₃,

(h) —CO₂R⁵⁷,

(i) hydroxy,

(j)—C(R⁵⁸)(R⁵⁹)—OH,

(k) —C₁₋₆alkyl-CO₂—R⁶⁰,

(l) C₁₋₆fluoroalkoxy,

(m) NO₂,

(n) NR⁶¹R⁶², and

(o) NHCOR⁶³;

R⁵³, R⁵⁴, R⁵⁵, R⁵⁶, R⁵⁷, R⁵⁸, R⁵⁹, R⁶⁰, R⁶¹, R⁶², R⁶³, are each independently chosen from the group consisting of:

(a) hydrogen, and

(b) C₁₋₆alkyl;

or R⁵⁴ and R⁵⁵, R⁵⁸ and R⁵⁹ or R⁶¹ and R⁶² together with the atom to which they are attached form a saturated monocyclic ring of 3, 4, 5, 6, or 7 atoms.

Materials that can serve as the cyclooxygenase-2 selective inhibitor of the present invention include diarylbenzopyran derivatives that are described in U.S. Pat. No. 6,340,694. Such diarylbenzopyran derivatives have the general formula shown below in formula XIV:

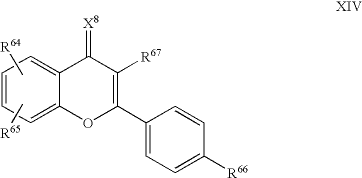

wherein:

X⁸ is an oxygen atom or a sulfur atom;

R⁶⁴ and R⁶⁵, identical to or different from each other, are independently a hydrogen atom, a halogen atom, a C₁-C₆ lower alkyl group, a trifluoromethyl group, an alkoxy group, a hydroxy group, a nitro group, a nitrile group, or a carboxyl group;

R⁶⁶ is a group of a formula: S(O)ₙR⁶⁸ wherein n is an integer of 0˜2, R⁶⁸ is a hydrogen atom, a C₁-C₆ lower alkyl group, or a group of a formula: NR⁶⁹ R⁷⁰ wherein R⁶⁹ and R⁷⁰, identical to or different from each other, are independently a hydrogen atom, or a C₁-C₆ lower alkyl group; and

R⁶⁷ is oxazolyl, benzo[b]thienyl, furanyl, thienyl, naphthyl, thiazolyl, indolyl, pyrolyl, benzofuranyl, pyrazolyl, pyrazolyl substituted with a C₁-C₆ lower alkyl group, indanyl, pyrazinyl, or a substituted group represented by the following structures:

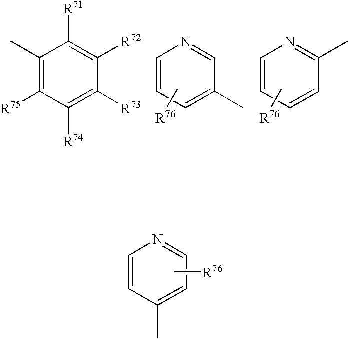

wherein:

R⁷¹ through R⁷⁵, identical to or different from one another, are independently a hydrogen atom, a halogen atom, a C₁-C₆ lower alkyl group, a trifluoromethyl group, an alkoxy group, a hydroxy group, a hydroxyalkyl group, a nitro group, a group of a formula: S(O)ₙR⁶⁸, a group of a formula: NR⁶⁹ R⁷⁰, a trifluoromethoxy group, a nitrile group a carboxyl group, an acetyl group, or a formyl group,

wherein n, R⁶⁸, R⁶⁹ and R⁷⁰ have the same meaning as defined by R⁶⁶ above; and

R⁷⁶ is a hydrogen atom, a halogen atom, a C₁-C₆ lower alkyl group, a trifluoromethyl group, an alkoxy group, a hydroxy group, a trifluoromethoxy group, a carboxyl group, or an acetyl group.

Materials that can serve as the cyclooxygenase-2 selective inhibitor of the present invention include 1-(4-sulfamylaryl)-3-substituted-5-aryl-2-pyrazolines that are described in U.S. Pat. No. 6,376,519. Such 1-(4-sulfamylaryl)-3-substituted-5-aryl-2-pyrazolines have the formula shown below in formula XV:

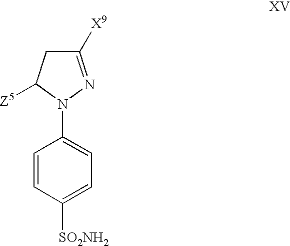

wherein:

X⁹ is selected from the group consisting of C₁-C₆ trihalomethyl, preferably trifluoromethyl; C₁-C₆ alkyl; and an optionally substituted or di-substituted phenyl group of formula XVI:

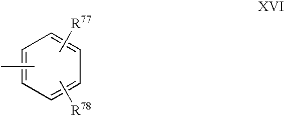

wherein:

R⁷⁷ and R⁷⁸ are independently selected from the group consisting of hydrogen, halogen, preferably chlorine, fluorine and bromine; hydroxyl; nitro; C₁-C₆ alkyl, preferably C₁-C₃ alkyl; C₁-C₆ alkoxy, preferably C₁-C₃ alkoxy; carboxy; C₁-C₆ trihaloalkyl, preferably trihalomethyl, most preferably trifluoromethyl; and cyano;

Z⁵ is selected from the group consisting of substituted and unsubstituted aryl.

Materials that can serve as the cyclooxygenase-2 selective inhibitor of the present invention include heterocycles that are described in U.S. Pat. No. 6,153,787. Such heterocycles have the general formulas shown below in formulas XVII and XVIII:

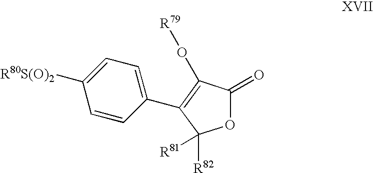

wherein:

R⁷⁹ is a mono-, di-, or tri-substituted C₁₋₁₂ alkyl, or a mono-, or an unsubstituted or mono-, di- or tri-substituted linear or branched C₂₋₁₀ alkenyl, or an unsubstituted or mono-, di- or tri-substituted linear or branched C₂₋₁₀ alkynyl, or an unsubstituted or mono-, di- or tri-substituted C₃₋₁₂ cycloalkenyl, or an unsubstituted or mono-, di- or tri-substituted C₅₋₁₂ cycloalkynyl, wherein the substituents are chosen from the group consisting of:

(a) halo, selected from F, Cl, Br, and I,

(b) OH,

(c) CF₃,

(d) C₃₋₆ cycloalkyl,

(e) ═O,

(f) dioxolane,

(g) CN; and

R⁸⁰ is selected from the group consisting of:

(a) CH₃,

(b) NH₂,

(c) NHC(O)CF₃,

(d) NHCH₃;

R⁸¹ and R⁸² are independently chosen from the group consisting of:

(a) hydrogen,

(b) C₁₋₁₀ alkyl;

or R⁸¹ and R⁸² together with the carbon to which they are attached form a saturated monocyclic carbon ring of 3, 4, 5, 6 or 7 atoms.

Formula XVIII is:

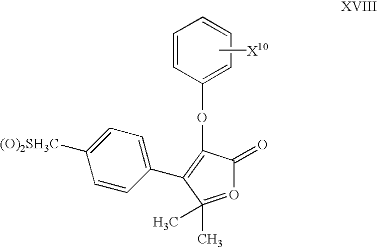

X¹⁰ is fluoro or chloro.

Materials that can serve as the cyclooxygenase-2 selective inhibitor of the present invention include 2,3,5-trisubstituted pyridines that are described in U.S. Pat. No. 6,046,217. Such pyridines have the general formula shown below in formula XIX:

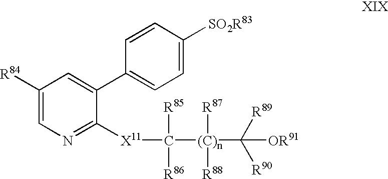

or a pharmaceutically acceptable salt thereof,

wherein:

X¹¹ is selected from the group consisting of:

(a) O,

(b) S,

(c) bond;

n is 0 or 1;

R⁸³ is selected from the group consisting of:

(a) CH₃,

(b) NH₂,

(c) NHC(O)CF₃;

R⁸⁴ is chosen from the group consisting of:

(a) halo,

(b) C₁₋₆ alkoxy,

(c) C₁₋₆ alkylthio,

(d) CN,

(e) C₁₋₆ alkyl,

(f) C₁₋₆ fluoroalkyl,

(g) N₃,

(h) —CO₂R⁹²,

(i) hydroxy,

(j) —C(R⁹³)(R⁹⁴)—OH,

(k) —C₁₋₆ alkyl-CO₂—R⁹⁵,

(i) C₁₋₆ fluoroalkoxy,

(m) NO₂,

(n) NR⁹⁶ R⁹⁷ ,

(o) NHCOR⁹⁸;

R⁸⁵ to R⁹⁸ are independantly chosen from the group consisting of

(a) hydrogen,

(b) C₁₋₆ alkyl;

or R⁸⁵ and R⁸⁹, or R⁸⁹ and R⁹⁰ together with the atoms to which they are attached form a carbocyclic ring of 3, 4, 5, 6 or 7 atoms, or R⁸⁵ and R⁸⁷ are joined to form a bond.

One preferred embodiment of the Cox-2 selective inhibitor of formula XIX is that wherein X is a bond.

Another preferred embodiment of the Cox-2 selective inhibitor of formula XIX is that wherein X is O.

Another preferred embodiment of the Cox-2 selective inhibitor of formula XIX is that wherein X is S.

Another preferred embodiment of the Cox-2 selective inhibitor of formula XIX is that wherein R⁸³ is CH₃.

Another preferred embodiment of the Cox-2 selective inhibitor of formula XIX is that wherein R⁸⁴ is halo or C₁₋₆ fluoroalkyl.

Materials that can serve as the cyclooxygenase-2 selective inhibitor of the present invention include diaryl bicyclic heterocycles that are described in U.S. Pat. No. 6,329,421. Such diaryl bicyclic heterocycles have the general formula shown below in formula XX:

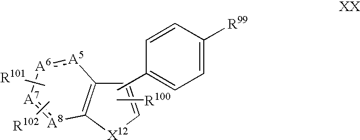

and pharmaceutically acceptable salts thereof wherein:

—A⁵═A⁶—A⁷═A⁸— is selected from the group consisting of:

(a) —CH═CH—CH═CH—,

(b) —CH₂—CH₂—CH₂—C(O)—, —CH₂—CH₂—C(O)—CH₂—, —CH₂—C(O)—CH₂—CH₂, —C(O)—CH₂—CH₂—CH₂,

(c) —CH₂—CH₂—C(O)—, —CH₂—C(O)—CH₂—, —C(O)—CH₂ —CH₂—

(d) —CH₂—CH₂—O—C(O)—, CH₂—O—C(O)—CH₂—, —O—C(O)—CH₂—CH₂—,

(e) —CH₂—CH₂—C(O)—O—, —CH₂—C(O)—OCH₂—, —C(O)—O—CH₂—CH₂—,

(f) —C(R¹⁰⁵)₂—O—C(O)—, —C(O)—O—C(R¹⁰⁵)₂—, —O—C(O)—C(R¹⁰⁵)₂—, —C(R¹⁰⁵)₂—C(O)—O—,

(g) —N═CH—CH═CH—,

(h) —CH═N—CH═CH—,

(i) —CH═CH—N═CH—,

(j) —CH═CH—CH═N—,

(k) —N═CH—CH═N—,

(l) —N═CH—N═CH—,

(m) —CH═N—CH═N—,

(n) —S—CH═N—,

(o) —S—N═CH—,

(p) —N═N—NH—,

(q) —CH═N—S—, and

(r)—N═CH—S—;

R⁹⁹ is selected from the group consisting of:

(a) S(O)₂CH₃,

(b) S(O)₂NH₂,

(c) S(O)₂NHCOCF₃,

(d) S(O)(NH)CH₃,

(e) S(O)(NH)NH₂,

(f) S(O)(NH)NHCOCF₃,

(g) P(O)(CH₃)OH, and

(h) P(O)(CH₃)NH₂;

R¹⁰⁰ is selected from the group consisting of:

(a) C₁₋₆ alkyl,

(b) C₃₋₇, cycloalkyl,

(c) mono- or di-substituted phenyl or naphthyl wherein the substituent is selected from the group consisting of:

(1) hydrogen,

(2) halo, including F, Cl, Br, I,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10) —CO₂—C₁₋₄ alkyl,

(11) —C(R¹⁰³)(R¹⁰⁴)—OH,

(12) —C(R¹⁰³)(R¹⁰⁴)—O—C₁₋₄ alkyl, and

(13) —C₁₋₆ alkyl-CO₂—R¹⁰⁶;

(d) mono- or di-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹⁰³)(R¹⁰⁴)—OH, and

(10) —C(R¹⁰³)(R¹⁰⁴)—O—C₁₋₄ alkyl;

(e) benzoheteroaryl which includes the benzo fused analogs of (d); R¹⁰¹ and R¹⁰² are the substituents residing on any position of —A⁵═A⁶—A⁷═A⁸—and are selected independently from the group consisting of:

(a) hydrogen,

(b) CF₃,

(c) CN,

(d) C₁₋₆ alkyl,

(e) —Q³ wherein Q³ is Q⁴, CO₂H, C(R¹⁰³)(R¹⁰⁴)OH,

(f) —O—Q⁴,

(g) —S—Q⁴, and

(h) optionally substituted:

(1) —C₁₋₅ alkyl-Q³,

(2) —O—C₁₋₅ alkyl-Q³,

(3) —S—C₁₅ alkyl-Q³,

(4) —C₁₋₃ alkyl-O—C₁₋₃ alkyl-Q³,

(5) —C₁₋₃ alkyl-S—C₁₋₃ alkyl-Q³,

(6) —C₁₋₅ alkyl-O—Q⁴,

(7) —C₁₋₅ alkyl-S—Q⁴,

wherein the substituent resides on the alkyl chain and the substituent is C₁₋₃ alkyl, and Q³ is Q₄, CO₂H, C(R¹⁰³)(R¹⁰⁴)OH Q⁴ is CO₂—C₁₋₄ alkyl, tetrazolyl-5-yl, or C(R¹⁰³)(R¹⁰⁴)O—C₁₋₄ alkyl;

R¹⁰³, R¹⁰⁴ and R¹⁰⁵ are each independently selected from the group consisting of

(a) hydrogen,

(b) C₁₋₆ alkyl; or

R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;

R¹⁰⁶ is hydrogen or C₁₋₆ alkyl;

R¹⁰⁷ is hydrogen, C₁₋₆ alkyl or aryl; X⁷ is O, S, NR¹⁰⁷, CO, C(R¹⁰⁷)₂, C(R¹⁰⁷)(OH), —C(R¹⁰⁷)═C(R¹⁰⁷)—; —C(R¹⁰⁷)═N—; —N═C(R¹⁰⁷)—.

Compounds that may act as cyclooxygenase-2 inhibitors include salts of 5-amino or a substituted amino 1,2,3-triazole compound that are described in U.S. Pat. No. 6,239,137. The salts are of a class of compounds of formula XXI:

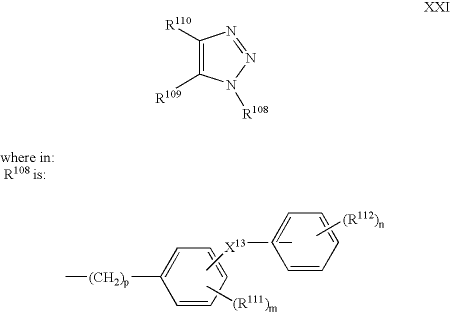

wherein:

p is 0 to 2; m is 0 to 4; and n is 0 to 5; X¹³ is O, S, SO, SO₂, CO, CHCN, CH₂ or C═NR¹¹³ where R¹¹³ is hydrogen, loweralkyl, hydroxy, loweralkoxy, amino, loweralkylamino, diloweralkylamino or cyano; and, R¹¹¹ and R¹¹² are independently halogen, cyano, trifluoromethyl, loweralkanoyl, nitro, loweralkyl, loweralkoxy, carboxy, lowercarbalkoxy, trifuloromethoxy, acetamido, loweralkylthio, loweralkylsulfinyl, loweralkylsulfonyl, trichlorovinyl, trifluoromethylthio, trifluoromethylsulfinyl, or trifluoromethylsulfonyl; R¹⁰⁹ is amino, mono or diloweralkyl amino, acetamido, acetimido, ureido, formamido, formamido or guanidino; and R¹¹⁰ is carbamoyl, cyano, carbazoyl, amidino or N-hydroxycarbamoyl; wherein the loweralkyl, loweralkyl containing, loweralkoxy and loweralkanoyl groups contain from 1 to 3 carbon atoms.

Materials that can serve as a cyclooxygenase-2 selective inhibitor of the present invention include pyrazole derivatives that are described in U.S. Pat. 6,136,831. Such pyrazole derivatives have the formula shown below in formula XXII:

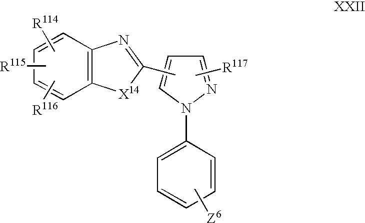

wherein:

R¹¹⁴ is hydrogen or halogen, R¹¹⁵ and R¹¹⁶ are each independently hydrogen, halogen, lower alkyl, lower alkoxy, hydroxy or lower alkanoyloxy;

R¹¹⁷ is lower haloalkyl or lower alkyl;

X¹⁴ is sulfur, oxygen or NH; and

Z⁶ is lower alkylthio, lower alkylsulfonyl or sulfamoyl; or a pharmaceutically acceptable salt thereof.

Materials that can serve as a cyclooxygenase-2 selective inhibitor of the present invention include substituted derivatives of benzosulphonamides that are described in U.S. Pat. No. 6,297,282. Such benzosulphonamide derivatives have the formula shown below in formula XXIII:

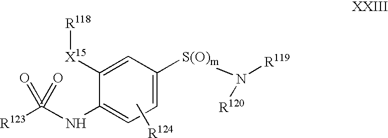

wherein:

X¹⁵ denotes oxygen, sulphur or NH;

R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;

R¹¹⁹ and R¹²⁰, independently from one another, denote hydrogen, an optionally polyfluorised alkyl group, an aralkyl, aryl or heteroaryl group or a group (CH₂)ₙ—X¹⁶; or

R¹¹⁹ and R¹²⁰, together with the N-atom, denote a 3 to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶; X¹⁶ denotes halogen, NO₂, —OR¹²¹, —COR¹²¹, —CO₂R¹²¹, —OCO₂R¹²¹, —CN, —CONR¹²¹OR¹²², —CONR¹²¹R¹²², —SR¹²¹, —S(O)R¹²¹, —S (O)₂R¹²¹—NR¹²¹R¹²², —NHC(O)R¹²¹, —NHS(O)₂R¹²¹; n denotes a whole number from 0 to 6;

R¹²³ denotes a straight-chained or branched alkyl group with 1-10 C-atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;

R¹²⁴ denotes halogen, hydroxy, a straight-chained or branched alkyl, alkoxy, acyloxy or alkyloxycarbonyl group with 1-6 C-atoms, which can optionally be mono- or polysubstituted by halogen, NO₂, —OR¹¹², —COR¹²¹, —CO₂R¹²¹, —OCO₂R¹²¹, —CN, —CONR¹²¹OR¹²², —CONR¹²¹R¹²², —SR¹²¹, —S(O)R¹²¹, —S(O)₂R¹²¹, —NR¹²¹R¹²², —NHC(O)R¹²¹, —NHS(O)₂R¹²¹, or a polyfluoroalkyl group;

R¹²¹ and R¹²², independently from one another, denote hydrogen, alkyl, aralkyl or aryl; and

m denotes a whole number from 0 to 2;

and the pharmaceutically-acceptable salts thereof.

Materials that can serve as a cyclooxygenase-2 selective inhibitor of the present invention include 3-phenyl-4-(4(methylsulfonyl)phenyl)-2-(5H)-furanones that are described in U.S. Pat. No. 6,239,173. Such 3-phenyl-4-(4(methylsulfonyl)phenyl)-2-(5H)-furanones have the formula shown below in formula XXIV:

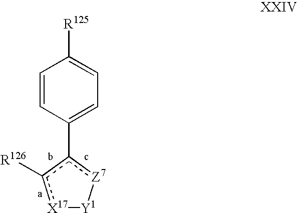

or pharmaceutically acceptable salts thereof wherein:

X¹⁷—Y¹—Z⁷-is selected from the group consisting of:

(a) —CH₂CH₂CH₂—,

(b) —C(O)CH₂CH₂—

(c) —CH₂CH₂C(O)—,

(d) —CR¹²⁹(R^{129′})—O—C(O)—,

(e) —C(O)—O—CR¹²⁹(R^{129′})—,

(f) —CH₂—NR¹²⁷—CH₂—

(g) —CR_{129 (R}^{129′})—NR¹²⁷ —C(O)—,

(h) —CR¹²⁸═CR^{128′}—S—,

(i) —S—CR¹²⁸═CR^{128′}—,

(j) —S—N═CH—,

(k) —CH═N—S—,

(l) —N═CR¹²⁸ —O—,

(m) —O—CR4═N—,

(n) —N═CR¹²⁸—NH—,

(o) —N═CR¹²⁸—S—, and

(p) —S—CR¹²⁸=N—,

(q) —C(O)—NR¹²⁷—CR¹²⁹(R^{129′})—,

(r) —R¹²⁷N—CH═CH— provided R₁₂₂ is not —S(O)₂CH₃,

(s) —CH═CH—NR¹²⁷— provided R¹²⁵ is not —S(O)₂CH₃,

when side b is a double bond, and sides a and c are single bonds; and

X¹⁷—Y¹—Z⁷-is selected from the group consisting of:

(a) ═CH—O—CH═, and

(b) ═CH—NR¹²⁷—CH═,

(c) ═N—S—CH═,

(d) ═CH—S—N═,

(e) ═N—O—CH═,

(f) ═CH—O—N═,

(g) ═N—S—N═,

(h) ═N—O—N═,

when sides a and c are double bonds and side b is a single bond;

R¹²⁵ is selected from the group consisting of:

(a) S(O)₂CH₃,

(b) S(O)₂NH₂,

(c) S(O)₂NHC(O)CF₃,

(d) S(O)(NH)CH₃,

(e) S(O)(NH)NH₂,

(f) S(O)(NH)NHC(O)CF₃,

(g) P(O)(CH₃)OH, and

(h) P(O)(CH₃)NH₂;

R¹²⁶ is selected from the group consisting of

(a) C₁₋₆ alkyl,

(b) C₃, C₄, C₅, C₆, and C₇, cycloalkyl,

(c) mono-, di- or tri-substituted phenyl or naphthyl,

wherein the substituent is selected from the group consisting of:

(1) hydrogen,

(2) halo,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10) —CO₂—C₁₋₄ alkyl,

(11) —C(R¹²⁹)(R¹³⁰)—OH,

(12) —C(R¹²⁹)(R¹³⁰)—O—C₁₋₄ alkyl, and

(13) —C₁₋₆ alkyl-CO₂—R¹²⁹;

(d) mono-, di- or tri-substituted heteroaryl wherein the heteroaryl is a monocyclic aromatic ring of 5 atoms, said ring having one hetero atom which is S, O, or N, and optionally 1, 2, or 3 additionally N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹²⁹)(R¹³⁰)—OH, and

(10) —C(R¹²⁹)(R¹³⁰)—O—C₁₋₄ alkyl;

(e) benzoheteroaryl which includes the benzo fused analogs of (d);

R¹²⁷ is selected from the group consisting of:

(a) hydrogen,

(b) CF₃,

(c) CN,

(d) C₁₋₆ alkyl,

(e) hydroxyC₁₋₆ alkyl,

(f) —C(O)—C₁₋₆ alkyl,

(g) optionally substituted:

(1) —C₁₋₅ alkyl-Q⁵,

(2) —C₁₋₃ alkyl-O—C₁₋₃ alkyl-Q⁵,

(3) —C ₁₋₃ alkyl-S—C₁₋₃ alkyl-Q⁵,

(4) —C₁₋₅ alkyl-O—Q⁵, or

(5) —C₁₋₅ alkyl-S—Q⁵,

wherein the substituent resides on the alkyl and the substituent is C₁₋₃ alkyl;

(h) —Q⁵;

R¹²⁸ and R^{128′} are each independently selected from the group consisting of:

(a) hydrogen,

(b) CF₃,

(c) CN,

(d) C₁₋₆ alkyl,

(e) —Q⁵,

(f) —O—Q⁵;

(g) —S—Q⁵, and

(h) optionally substituted:

(1) —C₁₋₅ alkyl-Q⁵,

(2) —O—C₁₋₅ alkyl-Q⁵,

(3) —S—C₁₋₅ alkyl-Q⁵,

(4) —C₁₋₃ alkyl-O—C₁₋₃ alkyl-Q⁵,

(5) —C₁₋₃ alkyl-S—C₁₋₃ alkyl-Q⁵,

(6) —C₁₋₅ alkyl-O—Q⁵,

(7) —C₁₋₅ alkyl-S—Q⁵,

wherein the substituent resides on the alkyl and the substituent is C₁₋₃ alkyl, and

R¹²⁹, R¹²⁹′, R¹³⁰R¹³¹ and R¹³² are each independently selected from the group consisting of:

(a) hydrogen,

(b) C₁₋₆ alkyl;

or R¹²⁹ and R¹³⁰ or R¹³¹ and R¹³² together with the carbon to which they are attached form a saturated monocyclic carbon ring of 3, 4, 5, 6 or 7 atoms,

Q5 is CO₂H, CO₂—C₁₋₄ alkyl, tetrazolyl-5-yl, C(R¹³¹)(R¹³²)(OH), or C(R¹³¹)(R¹³²)(O—C₁₋₄ alkyl);

provided that when X—Y—Z is —S—CR¹²⁸═CR^{128′}, then R¹²⁸ and R^{128′} are other than CF₃.

Materials that can serve as a cyclooxygenase-2 selective inhibitor of the present invention include bicycliccarbonyl indole compounds that are described in U.S. Pat. No. 6,303,628. Such bicycliccarbonyl indole compounds have the formula shown below in formula XXV:

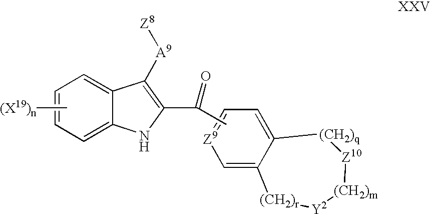

or the pharmaceutically acceptable salts thereof wherein

A⁹ is C₁₋₆ alkylene or —NR¹³³—;

z⁸ is C(═L³)R¹³⁴, or SO₂R¹³⁵;

Z⁹ is CH or N;

Z¹⁰ and Y² are independently selected from —CH₂—, O, S and —N—R¹³³;

m is 1, 2 or 3;

q and r are independently 0, 1 or 2;

X¹⁸ is independently selected from halogen, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di-(C₁₋₄ alkyl)amino and cyano;

n is 0, 1, 2, 3 or 4;

L³ is oxygen or sulfur;

R¹³³ is hydrogen or C₁₋₄ alkyl;

R¹³⁴ is hydroxy, C₁₋₆ alkyl, halo-substituted C₁₋₆ alkyl, C₁₋₆ alkoxy, halo-substituted C₁₋₆ alkoxy, C₃₋₇ cycloalkoxy, C₁₋₄ alkyl(C₃₋₇ cycloalkoxy), —NR¹³⁶ R¹³⁷, C₁₋₄ alkylphenyl-O— or phenyl-O—, said phenyl being optionally substituted with one to five substituents independently selected from halogen, C₁₋₄ alkyl, hydroxy, C₁₋₄ alkoxy and nitro; R¹³⁵ is C₁₋₆ alkyl or halo-substituted C₁₋₆ alkyl; and

R¹³⁶ and R¹³⁷ are independently selected from hydrogen, C₁₋₆ alkyl and halo-substituted C₁₋₆ alkyl.

Materials that can serve as a cyclooxygenase-2 selective inhibitor of the present invention include benzimidazole compounds that are described in U.S. Pat. No. 6,310,079. Such benzimidazole compounds have the formula shown below in formula XXVI:

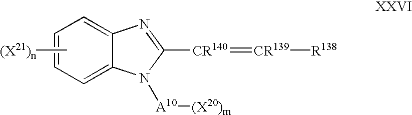

or a pharmaceutically acceptable salt thereof, wherein:

A¹⁰ is heteroaryl selected from a 5-membered monocyclic aromatic ring having one hetero atom selected from O, S and N and optionally containing one to three N atom(s) in addition to said hetero atom, or a 6-membered monocyclic aromatic ring having one N atom and optionally containing one to four N atom(s) in addition to said N atom; and said heteroaryl being connected to the nitrogen atom on the benzimidazole through a carbon atom on the heteroaryl ring;

X²⁰ is independently selected from halo, C₁-C₄ alkyl, hydroxy, C₁-C₄ alkoxy, halo-substituted C₁, -C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amonio, N-(C₁-C₄ alkyl)(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl; X²¹ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-(C₁-C₄ alkyl)-N-(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)cabonyl, cabamoyl, [N-(C₁-C₄ alkyl) amino]carbonyl, [N,N-di(C₁-C₄ alkyl)amino]carbonyl, N-carbomoylamino, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl;

R¹³⁸ is selected from hydrogen,

straight or branched C₁-C₄ alkyl optionally substituted with one to three substituent(s) wherein said substituents are independently selected from halo hydroxy, C₁-C₄ alkoxy, amino, N-(C₁-C₄ alkyl)amino and N,N-di(C₁-C₄ alkyl)amino,

C₃-C₈ cycloalkyl optionally substituted with one to three substituent(s) wherein said substituents are indepently selected from halo, C₁-C₄ alkyl, hydroxy, C₁-C₄ alkoxy, amino, N-(C₁-C₄ alkyl)amino and N, N-di(C₁-C₄ alkyl)amino,

C₄-C₈ cycloalkenyl optionally substituted with one to three substituent(s) wherein said substituents are independently selected from halo, C₁-C₄ alkyl, hydroxy, C₁-C₄ alkoxy, amino, N-(C₁-C₄ alkyl)amino and N,N-di(C₁-C₄ alkyl)amino,

phenyl optionally substituted with one to three substituent(s) wherein said substituents are independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-[C₁-C₄ alkyl)(C₁-C₄ alkanoyl)]amino, N-[(C₁-C₄ alkyl)sulfony]amino, N-[(halo-substituted C₁-C₄ alkyl)sulfonyl]amino, C₁-C₄ alkanoyl, carboxy, (C_{1 -C}₄ alkoxy)carbonyl, carbomoyl, [N-(C₁-C₄ alky)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl; and

heteroaryl selected from:

a 5-membered monocyclic aromatic ring having one hetero atom selected from O, S and N and optionally containing one to three N atom(s) in addition to said hetero atom; or a 6-membered monocyclic aromatic ring having one N atom and optionally containing one to four N atom(s) in addition to said N atom; and

said heteroaryl being optionally substituted with one to three substituent(s) selected from X²⁰;

R¹³⁹ and R¹⁴⁰ are independently selected from:

hydrogen,

halo,

C₁-C₄ alkyl,

phenyl optionally substituted with one to three substituent(s) wherein said substituents are independently selected from halo, C₁-C₄ alkyl, hydroxy, C₁-C₄ alkoxy, amino, N-(C₁-C₄ alkyl)amino and N,N-di(C₁ -C₄ alkyl)amino,

or R¹³⁸ and R¹³⁹ can form, together with the carbon atom to which they are attached, a C₃-C₇ cycloalkyl ring;

m is 0, 1, 2, 3, 4 or 5; and

n is 0, 1, 2, 3 or 4.

Materials that can serve as a cyclooxygenase-2 selective inhibitor of the present invention include indole compounds that are described in U.S. Pat. No. 6,300,363. Such indole compounds have the formula shown below in formula XXVII:

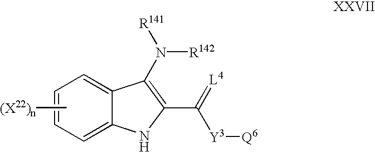

and the pharmaceutically acceptable salts thereof,

wherein:

L⁴ is oxygen or sulfur;

Y³ is a direct bond or C₁₋₄ alkylidene;

Q⁶ is:

(a) C₁₋₆ alkyl or halosubstituted C₁₋₆ alkyl, said alkyl being optionally substituted with up to three substituents independently selected from hydroxy, C₁₋₄ alkoxy, amino and mono- or di-(C₁₋₄ alkyl)amino,

(b) C₃₋₇ cycloalkyl optionally substituted with up to three substituents independently selected from hydroxy, C₁₋₄ alkyl and C₁₋₄ alkoxy,

(c) phenyl or naphthyl, said phenyl or naphthyl being optionally substituted with up to four substituents independently selected from: (c-1) halo, C₁₋₄ alkyl, halosubstituted C₁₋₄ alkyl, hydroxy, C₁₋₄ alkoxy, halosubstituted C₁₋₄ alkoxy, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OH, C₁₋₄ alkyl-OR¹⁴³, CONH₂, CONH(C₁₋₄ alkyl), CON(C₁₋₄ alkyl)₂ and —O—Y-phenyl, said phenyl being optionally substituted with one or two substituents independently selected from halo, C₁₋₄ alkyl, CF₃, hydroxy, OR¹⁴³, S(O)ₘR¹⁴³, amino, mono- or di-(C₁₋₄ alkyl)amino and CN;

(d) a monocyclic aromatic group of 5 atoms, said aromatic group having one heteroatom selected from O, S and N and optionally containing up to three N atoms in addition to said heteroatom, and said aromatic group being substituted with up to three substitutents independently selected from:

(d-1) halo, C₁₋₄ alkyl, halosubstituted C₁₋₄ alkyl, hydroxy, C₁₋₄ alkoxy, halosubstituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³;

(e) a monocyclic aromatic group of 6 atoms, said aromatic group having one heteroatom which is N and optionally containing up to three atoms in addition to said heteroatom, and said aromatic group being substituted with up to three substituents independently selected from the above group (d-1);

R¹⁴¹ is hydrogen or C₁₋₆ alkyl optionally substituted with a substituent selected independently from hydroxy, OR¹⁴³, nitro, amino, mono- or di-(C₁₋₄ alkyl)amino, CO₂H, CO₂(C₁₋₄ alkyl), CONH₂, CONH(C₁₋₄ alkyl) and CON(C₁₋₄ alkyl)₂;

R¹⁴² is:

(a) hydrogen,

(b) C₁₋₄ alkyl,

(c) C(O)R¹⁴⁵,

wherein R¹⁴⁵ is selected from:

(c-1) C₁₋₂₂ alkyl or C₂₋₂₂ alkenyl, said alkyl or alkenyl being optionally substituted with up to four substituents independently selected from: (c-1-1) halo, hydroxy, OR¹⁴³, S(O)ₘR¹⁴³, nitro, amino, mono- or di-(C₁₋₄ alkyl)amino, NHSO₂R¹⁴³, CO₂H, CO₂(C₁₋₄ alkyl), CONH₂, CONH(C₁₋₄ alkyl), CON(C₁₋₄ alkyl)₂, OC(O)R¹⁴³, thienyl, naphthyl and groups of the following formulae:

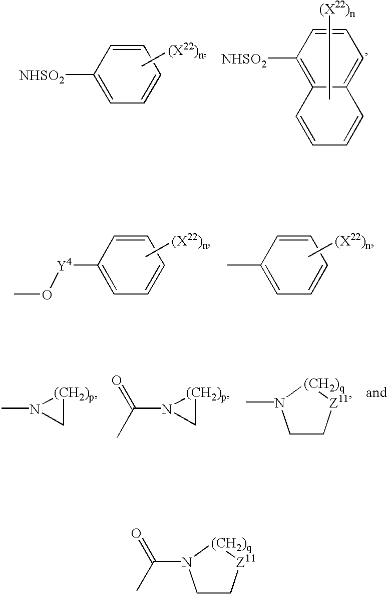

(c-2) C₁₋₂₂ alkyl or C₂₋₂₂ alkenyl, said alkyl or alkenyl being optionally substituted with five to forty-five halogen atoms,

(c-3) —Y⁵—C₃₋₇ cycloalkyl or —Y⁵—C₃₋₇ cycloalkenyl, said cycloalkyl or cycloalkenyl being optionally substituted with up to three substituent independently selected from:

(c-3-1) C₁₋₄ alkyl, hydroxy, OR¹⁴³, S(O)ₘR¹⁴³, amino, mono- or di-(C₁₋₄ alkyl)amino, CONH₂, CONH(C₁₋₄ alkyl) and CON(C₁₋₄ alkyl)₂, (c-4) phenyl or naphthyl, said phenyl or naphthyl being optionally substituted with up to seven (preferably up to seven) substituents independently selected from:

(c-4-1) halo, C₁₋₈ alkyl, C₁₋₄ alkyl-OH, hydroxy, C₁₋₈ alkoxy, halosubstituted C₁₋₈ alkyl, halosubstituted C₁₋₈ alkoxy, CN, nitro, S(O)ₘR¹⁴³, SO₂NH₂, SO₂NH(C₁₋₄ alkyl), SO₂N(C₁₋₄ alkyl)₂, amino, C₁₋₄ alkylamino, di-(C₁₋₄ alkyl)amino, CONH₂, CONH(C₁₋₄ alkyl), CON(C₁₋₄ alkyl)₂, OC(O)R¹⁴³, and phenyl optionally substituted with up to three substituents independently selected from halo, C₁₋₄ alkyl, hydroxy, OCH₃, CF₃, OCF₃, CN, nitro, amino, mono- or di-(C₁₋₄ alkyl)amino, CO₂H, CO₂(C₁₋₄ alkyl) and CONH₂,

(c-5) a monocyclic aromatic group as defined in (d) and (e) above, said aromatic group being optionally substituted with up to three substituents independently selected from:

(c-5-1) halo, C₁₋₈ alkyl, C₁₋₄ alkyl-OH, hydroxy, C₁₋₈ alkoxy, CF₃, OCF₃, CN, nitro, S(O)ₘR¹⁴³, amino, mono- or di-(C₁₋₄ alkyl)amino, CONH₂, CONH(C₁₋₄ alkyl), CON(C₁₋₄ alkyl)₂, CO₂H and CO₂(C₁₋₄ alkyl), and —Y— phenyl, said phenyl being optionally substituted with up to three substituents independently selected halogen, C₁₋₄ alkyl, hydroxy, C₁₋₄ alkoxy, CF₃, OCF₃, CN, nitro, S(O)ₘR¹⁴³, amino, mono- or di-(C₁₋₄ alkyl)amino, CO₂H, CO₂(C₁₋₄ alkyl), CONH₂, CONH(C₁₋₄ alkyl) and CON(C₁₋₄ alkyl)₂,

(c-6) a group of the following formula:

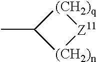

X²² is halo, C₁₋₄ alkyl, hydroxy, C₁₋₄ alkoxy, halosubstitutued C₁₋₄ alkoxy, S(O)ₘR¹⁴³, amino, mono- or di-(C₁₋₄ alkyl)amino, NHSO₂R¹⁴³, nitro, halosubstitutued C₁₋₄ alkyl, CN, CO₂H, CO₂(C₁₋₄ alkyl), C₁₋₄ alkyl-OH, C₁₋₄ alkylOR¹⁴³, CONH₂, CONH(C₁₋₄ alkyl) or CON(C₁₋₄ alkyl)₂; R¹⁴³ is C₁₋₄ alkyl or halosubstituted C₁₋₄ alkyl;

m is 0, 1 or 2; n is 0, 1, 2 or 3; p is 1, 2, 3, 4 or 5; q is 2 or 3; Z¹¹ is oxygen, sulfur or NR¹⁴⁴; and p1 R¹⁴⁴ is hydrogen, C₁₋₆ alkyl, halosubstitutued C₁₋₄ alkyl or —Y⁵-phenyl, said phenyl being optionally substituted with up to two substituents independently selected from halo, C₁₋₄ alkyl, hydroxy, C₁₋₄ alkoxy, S(O)ₘR¹⁴³, amino, mono- or di-(C₁₋₄ alkyl)amino, CF₃, OCF₃, CN and nitro;

with the proviso that a group of formula —Y⁵—Q is not methyl or ethyl when X²² is hydrogen;

L⁴ is oxygen;

R¹⁴¹ is hydrogen; and

R142 is acetyl.

Materials that can serve as a cyclooxygenase-2 selective inhibitor of the present invention include aryl phenylhydrazides that are described in U.S. Pat. No. 6,077,869. Such aryl phenylhydrazides have the formula shown below in formula XXVIII:

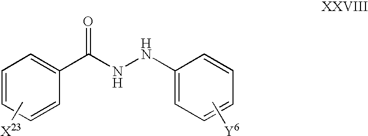

wherein:

X²³ and Y⁶ are selected from hydrogen, halogen, alkyl, nitro, amino or other oxygen and sulfur containing functional groups such as hydroxy, methoxy and methylsulfonyl.

Materials that can serve as a cyclooxygenase-2 selective inhibitor of the present invention include 2-aryloxy, 4-aryl furan-2-ones that are described in U.S. Pat. No. 6,140,515. Such 2-aryloxy, 4-aryl furan-2-ones have the. formula shown below in formula XXIX:

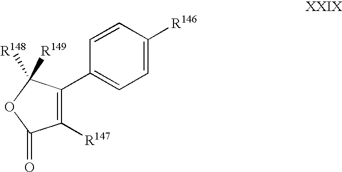

or a pharmaceutical salt thereof,

wherein:

R¹⁴⁶ is selected from the group consisting of SCH₃, —S(O)₂CH₃ and —S(O)₂NH₂;

R¹⁴⁷ is selected from the group consisting of OR¹⁵⁰, mono or di-substituted phenyl or pyridyl wherein the substituents are selected from the group consisting of methyl, chloro and F;

R¹⁵⁰ is unsubstituted or mono or di-substituted phenyl or pyridyl wherein the substituents are selected from the group consisting of methyl, chloro and F;

R¹⁴⁸ is H, C₁₋₄ alkyl optionally substituted with 1 to 3 groups of F, Cl or Br; and

R¹⁴⁹ is H, C₁₋₄ alkyl optionally substituted with 1 to 3 groups of F, Cl or Br, with the proviso that R¹⁴⁸ and R¹⁴⁹ are not the same.

Materials that can serve as a cyclooxygenase-2 selective inhibitor of the present invention include bisaryl compounds that are described in U.S. Pat. No. 5,994,379. Such bisaryl compounds have the formula shown below in formula XXX:

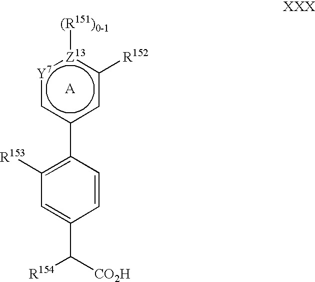

or a pharmaceutically acceptable salt, ester or tautomer thereof,

wherein:

Z¹³ is C or N;

when Z¹³ is N, R¹⁵¹ represents H or is absent, or is taken in conjunction with R¹⁵² as described below:

when Z¹³ is C, R¹⁵¹ represents H and R¹⁵² is a moiety which has the following characteristics:

(a) it is a linear chain of 3-4 atoms containing 0-2 double bonds, which can adopt an energetically stable transoid configuration and if a double bond is present, the bond is in the trans configuration,

(b) it is lipophilic except for the atom bonded directly to ring A, which is either lipophilic or non-lipophilic, and

(c) there exists an energetically stable configuration planar with ring A to within about 15 degrees;

or R¹⁵¹ and R¹⁵² are taken in combination and represent a 5- or 6-membered aromatic or non-aromatic ring D fused to ring A, said ring D containing 0-3 heteroatoms selected from O, S and N;

said ring D being lipophilic except for the atoms attached directly to ring A, which are lipophilic or non-lipophilic, and said ring D having available an energetically stable configuration planar with ring A to within about 15 degrees;

said ring D further being substituted with 1 R^{a} group selected from the group consisting of: C₁₋₂ alkyl, —OC₁₋₂ alkyl, —NHC₁₋₂ alkyl, —N(C₁₋₂ alkyl)₂, —C(O)C₁₋₂ alkyl, —S—C₁₋₂ alkyl and —C(S)C₁₋₂ alkyl;

Y⁷ represents N, CH or C—OC₁₋₃ alkyl, and when Z¹³ is N, Y⁷ can also represent a carbonyl group;

R¹⁵³ represents H, Br, Cl or F; and

R¹⁵⁴ represents H or CH₃.

Materials that can serve as a cyclooxygenase-2 selective inhibitor of the present invention include 1,5-diarylpyrazoles that are described in U.S. Pat. No. 6,028,202. Such 1,5-diarylpyrazoles have the formula shown below in formula XXXI:

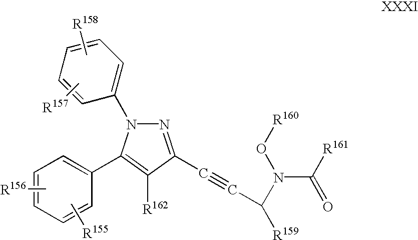

wherein:

R¹⁵⁵, R¹⁵⁶, R¹⁵⁷, and R¹⁵⁸ are independently selected from the groups consisting of hydrogen, C₁₋₅ alkyl, C₁₋₅ alkoxy, phenyl, halo, hydroxy, C₁₋₅ alkylsulfonyl, C₁₋₅ alkylthio, trihaloC₁₋₅ alkyl, amino, nitro and 2-quinolinylmethoxy;

R¹⁵⁹ is hydrogen, C₁₋₅ alkyl, trihaloC₁₋₅ alkyl, phenyl, substituted phenyl where the phenyl substitutents are halogen, C₁₋₅ alkoxy, trihaloC₁₋₅ alkyl or nitro or R¹⁵⁹ is heteroaryl of 5-7 ring members where at least one of the ring members is nitrogen, sulfur or oxygen;

R¹⁶⁰ is hydrogen, C₁₋₅ alkyl, phenyl C₁₋₅ alkyl, substituted phenyl C₁₋₅ alkyl where the phenyl substitutents are halogen, C₁₋₅ alkoxy, trihaloC₁₋₅ alkyl or nitro, or R¹⁶⁰ is C₁₋₅ alkoxycarbonyl, phenoxycarbonyl, substituted phenoxycarbonyl where the phenyl substitutents are halogen, C₁₋₅ alkoxy, trihaloC₁₋₅ alkyl or nitro;

R¹⁶¹ is C₁₋₁₀ alkyl, substituted C₁₋₁₀ alkyl where the substituents are halogen, trihaloC₁₋₅ alkyl, C₁₋₅ alkoxy, carboxy, C₁₋₅ alkoxycarbonyl, amino, C₁₋₅ alkylamino, diC₁₋₅ alkylamino, diC₁₋₅ alkylaminoC₁₋₅ alkylamino, C₁₋₅ alkylaminoC₁₋₅ alkylamino or a heterocycle containing 4-8 ring atoms where one more of the ring atoms is nitrogen, oxygen or sulfur, where said heterocycle may be optionally substituted with C₁₋₅ alkyl; or R¹⁶¹ is phenyl, substituted phenyl (where the phenyl substitutents are one or more of C₁₋₅ alkyl, halogen, C₁₋₅ alkoxy, trihaloC₁₋₅ alkyl or nitro), or R¹⁶¹ is heteroaryl having 5-7 ring atoms where one or more atoms are nitrogen, oxygen or sulfur, fused heteroaryl where one or more 5-7 membered aromatic rings are fused to the heteroaryl; or

R¹⁶¹ is NR¹⁶³R¹⁶⁴ where R¹⁶³ and R¹⁶⁴ are independently selected from hydrogen and C₁₋₅ alkyl or R¹⁶³ and R¹⁶⁴ may be taken together with the depicted nitrogen to form a heteroaryl ring of 5-7 ring members where one or more of the ring members is nitrogen, sulfur or oxygen where said heteroaryl ring may be optionally substituted with C₁₋₅ alkyl;

R¹⁶² is hydrogen, C₁₋₅ alkyl, nitro, amino, and halogen;

and pharmaceutically acceptable salts thereof.

Materials that can serve as a cyclooxygenase-2 selective inhibitor of the present invention include 2-substituted imidazoles that are described in U.S. Pat. No. 6,040,320. Such 2-substituted imidazoles have the formula shown below in formula XXXII:

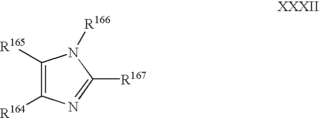

wherein:

R¹⁶⁴ is phenyl, heteroaryl wherein the heteroaryl contains 5 to 6 ring atoms, or

substituted phenyl;

wherein the substituents are independently selected from one or members of the group consisting of C₁₋₅ alkyl, halogen, nitro, trifluoromethyl and nitrile;

R¹⁶⁵ is phenyl, heteroaryl wherein the heteroaryl contains 5 to 6 ring atoms,

substituted heteroaryl;

wherein the substituents are independently selected from one or more members of the group consisting of C₁₋₅ alkyl and halogen, or substituted phenyl,

wherein the substituents are independently selected from one or members of the group consisting of C₁₋₅ alkyl, halogen, nitro, trifluoromethyl and nitrile;

R¹⁶⁶ is hydrogen, SEM, C₁₋₅ alkoxycarbonyl, aryloxycarbonyl, arylC₁₋₅ alkyloxycarbonyl, arylC₁₋₅alkyl, phthalimidoC₁₋₅ alkyl, aminoC₁₋₅ alkyl, diaminoC₁₋₅ alkyl, succinimidoC₁₋₅ alkyl, C₁₋₅ alkylcarbonyl, arylcarbonyl, C₁₋₅ alkylcarbonylC₁₋₅ alkyl, aryloxycarbonylC₁₋₅ alkyl, heteroarylC₁₋₅ alkyl where the heteroaryl contains 5 to 6 ring atoms, or substituted arylC₁₋₅alkyl,

wherein the aryl substituents are independently selected from one or more members of the group consisting of C₁₋₅ alkyl, C₁₋₅ alkoxy, halogen, amino, C₁₋₅ alkylamino, and diC₁₋₅ alkylamino;

R¹⁶⁷ is (A¹¹)ₙ-(CH¹⁶⁵)_{q}—X²⁴ wherein:

A¹¹ is sulfur or carbonyl;

n is 0 or 1;

q is 0-9;

X²⁴ is selected from the group consisting of hydrogen, hydroxy, halogen, vinyl, ethynyl, C₁₋₅ alkyl, C₃₋₇ cycloalkyl, C₁₋₅ alkoxy, phenoxy, phenyl, arylC₁₋₅ alkyl, amino, C₁₋₅ alkylamino, nitrile, phthalimido, amido, phenylcarbonyl, C₁₋₅ alkylaminocarbonyl, phenylaminocarbonyl, arylC₁₋₅ alkylaminocarbonyl, C₁₋₅ alkylthio, C₁₋₅ alkylsulfonyl, phenylsulfonyl,

substituted sulfonamido,

wherein the sulfonyl substituent is selected from the group consisting of C₁₋₅ alkyl, phenyl, araC₁₋₅ alkyl, thienyl, furanyl, and naphthyl;

substituted vinyl,

wherein the substituents are independently selected from one or members of the group consisting of fluorine, bromine, chlorine and iodine,

substituted ethynyl,

wherein the substituents are independently selected from one or more members of the group consisting of fluorine, bromine chlorine and iodine,

substituted C₁₋₅ alkyl,

wherein the substituents are selected from the group consisting of one or more C₁₋₅ alkoxy, trihaloalkyl, phthalimido and amino,

substituted phenyl,

wherein the phenyl substituents are independently selected from one or more members of the group consisting of C₁₋₅ alkyl, halogen and C₁₋₅ alkoxy,

substituted phenoxy,

wherein the phenyl substituents are independently selected from one or more members of the group consisting of C₁₋₅ alkyl, halogen and C₁₋₅ alkoxy,

substituted C₁₋₅ alkoxy, wherein the alkyl substituent is selected from the group consisting of phthalimido and amino,

substituted arylC₁₋₅ alkyl,

wherein the alkyl substituent is hydroxyl,

substituted arylC₁₋₅ alkyl,

wherein the phenyl substituents are independently selected from one or more members of the group consisting of C₁₋₅ alkyl, halogen and C₁₋₅ alkoxy,

substituted amido,

wherein the carbonyl substituent is selected from the group consisting of C₁₋₅ alkyl, phenyl, arylC₁₋₅ alkyl, thienyl, furanyl, and naphthyl,

substituted phenylcarbonyl,

wherein the phenyl substituents are independently selected from one or members of the group consisting of C₁₋₅ alkyl, halogen and C₁₋₅ alkoxy,

substituted C₁₋₅ alkylthio,

wherein the alkyl substituent is selected from the group consisting of hydroxy and phthalimido,

substituted C₁₋₅ alkylsulfonyl,

wherein the alkyl substituent is selected from the group consisting of hydroxy and phthalimido,

substituted phenylsulfonyl,

wherein the phenyl substituents are independently selected from one or members of the group consisting of bromine, fluorine, chlorine, C₁₋₅ alkoxy and trifluoromethyl,

with the proviso:

if A¹¹ is sulfur and X²⁴ is other than hydrogen, C₁₋₅ alkylaminocarbonyl, phenylaminocarbonyl, arylC₁₋₅ alkylaminocarbonyl, C₁₋₅ alkylsulfonyl or phenylsulfonyl, then q must be equal to or greater than 1;

if A¹¹ is sulfur and q is 1, then X²⁴ cannot be C₁₋₂ alkyl;

if A¹¹ is carbonyl and q is 0, then X24 cannot be vinyl, ethynyl, C₁₋₅ alkylaminocarbonyl, phenylaminocarbonyl, arylC₁₋₅ alkylaminocarbonyl,C₁₋₅ alkylsulfonyl or phenylsulfonyl;

if A¹¹ is carbonyl, q is 0 and X²⁴ is H, then R¹⁶⁶ is not SEM (2-(trimethylsilyl)ethoxymethyl);

if n is 0 and q is 0, then X²⁴ cannot be hydrogen;

and pharmaceutically acceptable salts thereof.

Materials that can serve as a cyclooxygenase-2 selective inhibitor of the present invention include 1,3- and 2,3-diarylcycloalkano and cycloalkeno pyrazoles that are described in U.S. Pat. No. 6,083,969. Such 1,3- and 2,3-diarylpyrazole compounds have the general formulas shown below in formulas XXXIII and XXXIV:

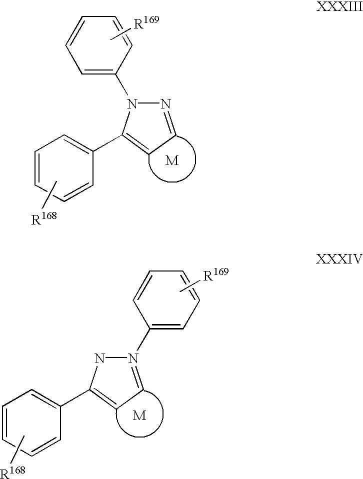

wherein:

R¹⁶⁸ and R¹⁶⁹ are independently selected from the group consisting of hydrogen, halogen, (C₁-C₆)alkyl, (C₁-C₆)alkoxy, nitro, amino, hydroxy, trifluoro, —S(C₁-C₆)alkyl, —SO(C₁-C₆)alkyl and —SO₂(C₁-C₆)alkyl; and the fused moiety M is a group selected from the group consisting of an optionally substituted cyclohexyl and cycloheptyl group having the formulae:

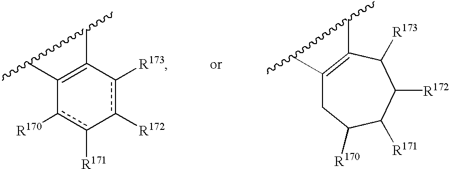

wherein:

R¹⁷⁰ is selected from the group consisting of hydrogen, halogen, hydroxy and carbonyl;

or R¹⁷⁰ and R¹⁷¹ taken together form a moiety selected from the group consisting of —OCOCH₂—, —ONH(CH₃)COCH₂—, —OCOCH.dbd. and —O—;

R¹⁷¹ and R¹⁷² are independently selected from the group consisting of hydrogen, halogen, hydroxy, carbonyl, amino, (C₁-C₆)alkyl, (C₁-C₆)alkoxy, ═NOH, —NR¹⁷⁴R¹⁷⁵, —OCH₃, —OCH₂CH₃, —OSO₂NHCO₂CH₃, ═CHCO₂CH₂CH₃, —CH₂CO₂H, —CH₂CO₂CH₃, —CH₂CO₂ CH₂CH₃, —CH₂CON(CH₃)₂, —CH₂CO₂NHCH₃, —CHCHCO₂CH₂CH₃, —OCON(CH₃)OH, —C(COCH₃)₂, di(C₁-C₆)alkyl and di(C₁-C₆)alkoxy;

R¹⁷³ is selected from the group consisting of hydrogen, halogen, hydroxy, carbonyl, amino, (C₁-C₆)alkyl, (C₁-C₆)alkoxy and optionally substituted carboxyphenyl, wherein substituents on the carboxyphenyl group are selected from the group consisting of halogen, hydroxy, amino, (C₁-C₆)alkyl and (C₁-C₆)alkoxy;

or R¹⁷² and R¹⁷³ taken together form a moiety selected from the group consisting of —O— and

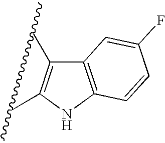

Ris selected from the group consisting of hydrogen, OH, —OCOCH₃, —COCH₃ and (C₁-C₆)alkyl; and

R¹⁷⁵ is selected from the group consisting of hydrogen, OH, —OCOCH₃, —COCH₃, (C₁-C₆)alkyl, —CONH₂ and —SO₂CH₃; with the proviso that

if M is a cyclohexyl group, then R¹⁷⁰ through R¹⁷³ may not all be hydrogen; and

pharmaceutically acceptable salts, esters and pro-drug forms thereof.

Materials that can serve as a cyclooxygenase-2 selective inhibitor of the present invention include esters derived from indolealkanols and novel amides derived from indolealkylamides that are described in U.S. Pat. No. 6,306,890. Such compounds have the general formula shown below in formula XXXV:

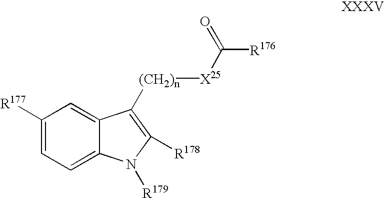

wherein:

R¹⁷⁶ is C₁ to C₆ alkyl, C₁ to C₆ branched alkyl, C₄ to C₈ cycloalkyl, C₁ to C₆ hydroxyalkyl, branched C₁ to C₆ hydroxyalkyl, hydroxy substituted C₄ to C₈ aryl, primary, secondary or tertiary C₁ to C₆ alkylamino, primary, secondary or tertiary branched C₁ to C₆ alkylamino, primary, secondary or tertiary C₄ to C₈ arylamino, C₁ to C₆ alkylcarboxylic acid, branched C₁ to C₆ alkylcarboxylic acid, C₁ to C₆ alkylester, branched C₁ to C₆ alkylester, C₄ to C₈ aryl, C₄ to C₈ arylcarboxylic acid, C₄ to C₈ arylester, C₄ to C₈ aryl substituted C₁ to C₆ alkyl, C₄ to C₈ heterocyclic alkyl or aryl with O, N or S in the ring, alkyl-substituted or aryl-substituted C₄ to C₈ heterocyclic alkyl or aryl with O, N or S in the ring, or halo-substituted versions thereof, where halo is chloro, bromo, fluoro or iodo,

R¹⁷⁷ is C₁ to C₆ alkyl, C₁ to C₆ branched alkyl, C₄ to C₈ cycloalkyl, C₄ to C₈ aryl, C₄ to C₈ aryl-substituted C₁ to C₆ alkyl, C₁ to C₆ alkoxy, C₁ to C₆ branched alkoxy, C₄ to C₈ aryloxy, or halo-substituted versions thereof or R¹⁷⁷ is halo where halo is chloro, fluoro, bromo, or iodo;

R¹⁷⁸ is hydrogen, C₁ to C₆ alkyl or C₁ to C₆ branched alkyl;

R¹⁷⁹ is C₁ to C₆ alkyl, C₄ to C₈ aroyl, C₄ to C₈ aryl, C₄ to C₈ heterocyclic alkyl or aryl with O, N or S in the ring, C₄ to C₈ aryl-substituted C₁ to C₆ alkyl, alkyl-substituted or aryl-substituted C₄ to C₈ heterocyclic alkyl or aryl with O, N or S in the ring, alkyl-substituted C₄ to C₈ aroyl, or alkyl-substituted C₄ to C₈ aryl, or halo-substituted versions thereof where halo is chloro, bromo, or iodo;

n is 1, 2, 3, or 4; and

X²⁵ is O, NH, or N—R¹⁸⁰, where R¹⁸⁰ is C₁ to C₆ alkyl or C₁ to C₆ branched alkyl.

Materials that can serve as a cyclooxygenase-2 selective inhibitor of the present invention include pyridazinone compounds that are described in U.S. Pat. No. 6,307,047. Such pyridazinone compounds have the formula shown below in formula XXXVI:

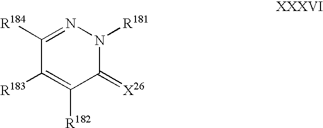

or a pharmaceutically acceptable salt, ester, or prodrug thereof,

wherein:

X²⁶ is selected from the group consisting of O, S, —NR¹⁸⁵, —NOR^{a}, and —NNR^{b}R^{c};

R¹⁸⁵ is selected from the group consisting of alkenyl, alkyl, aryl, arylalkyl, cycloalkenyl, cycloalkenylalkyl, cycloalkyl, cycloalkylalkyl, heterocyclic, and heterocyclic alkyl;

R^{a}, R^{b}, and R^{c} are independently selected from the group consisting of alkyl, aryl, arylalkyl, cycloalkyl, and cycloalkylalkyl;

R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙC(O)R¹⁸⁶, —(CH₂)ₙCH(OH)R¹⁸⁶,—(CH₂)ₙC(NOR^{d})R¹⁸⁶, —(CH₂)ₙCH(NOR^{d})R¹⁸⁶, —(CH₂)ₙCH(NR^{d}R^{e})R¹⁸⁶, —R¹⁸⁷ R¹⁸⁸, —(CH₂)ₙC□CR¹⁸⁸, —(CH₂)ₙ[CH(CX²⁶′₃)]ₘ(CH₂)ₚR¹⁸⁸, —(CH₂)ₙ(CX²⁶′₂)ₘ(CH₂)ₚR¹⁸⁸, and —(CH₂)ₙ(CHX²⁶′)ₘ(CH₂)ₘR¹⁸⁸;

R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;

R¹⁸⁷ is selected from the group consisting of alkenylene, alkylene, halo-substituted alkenylene, and halo-substituted alkylene;

R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;

R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl;

X^{26′} is halogen;

m is an integer from 0-5;

n is an integer from 0-10; and

p is an integer from 0-10; and

R¹⁸², R¹⁸³, and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸, and Z¹⁴;

provided that one of R¹⁸², R¹⁸³, or R¹⁸⁴ must be Z¹⁴ , and further provided that only one of R¹⁸², R¹⁸³, or R¹⁸⁴ is Z¹⁴;

Z¹⁴ is selected from the group consisting of:

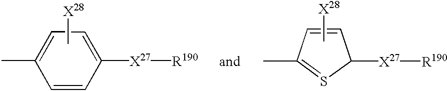

²⁷ is selected from the group consisting of S(O)₂, S(O)(NR¹⁹¹), S(O), Se(O)₂, P(O)(OR¹⁹²), and P(O)(NR¹⁹³R¹⁹⁴);

X²⁸ is selected from the group consisting of hydrogen, alkenyl, alkyl, alkynyl and halogen;

R¹⁹⁰ is selected from the group consisting of alkenyl, alkoxy, alkyl, alkylamino, alkylcarbonylamino, alkynyl, amino, cycloalkenyl, cycloalkyl, dialkylamino, —NHNH₂, and —NCHN(R¹⁹¹)R¹⁹²;

R¹⁹¹, R¹⁹², R¹⁹³, and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing 1 or 2 heteroatoms selected from the group consisting of O, S, and NR¹⁸⁸;

Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, —NC(R¹⁹⁷)R¹⁹⁵, and —N(R¹⁹⁷)R¹⁹⁵;

R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl, and NR¹⁹⁹R²⁰⁰; and

R¹⁹⁷, R¹⁹⁸, R¹⁹⁹, and R²⁰⁰ are independently selected from the group consisting of hydrogen, alkenyl, alkoxy, alkyl, cycloalkenyl, cycloalkyl, aryl, arylalkyl, heterocyclic, and heterocyclic alkyl.

Materials that can serve as a cyclooxygenase-2 selective inhibitor of the present invention include benzosulphonamide derivatives that are described in U.S. Pat. No. 6,004,948. Such benzosulphonamide derivatives have the formula shown below in formula XXXVII:

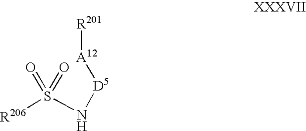

herein:

A¹² denotes oxygen, sulphur or NH;

R²⁰¹ denotes a cycloalkyl, aryl or heteroaryl group optionally mono-or polysubstituted by halogen, alkyl, CF₃ or alkoxy;

D⁵ denotes a group of formula XXXVIII or XXXIX:

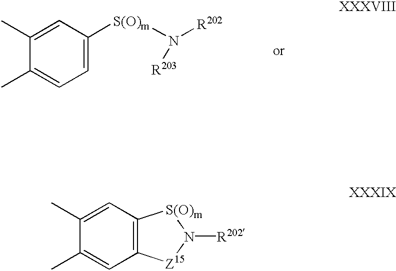

R²⁰² and R²⁰³ independently of each other denote hydrogen, an optionally polyfluorinated alkyl radical, an aralkyl, aryl or heteroaryl radical or a radical (CH₂)ₙ—X²⁹; or

R²⁰² and R²⁰³ together with the N-atom denote a three- to seven-membered, saturated, partially or totally unsaturated heterocycle with one or more heteroatoms N, O, or S, which may optionally be substituted by oxo, an alkyl, alkylaryl or aryl group or a group (CH₂)ₙ—X²⁹, R²⁰²′ denotes hydrogen, an optionally polyfluorinated alkyl group, an aralkyl, aryl or heteroaryl group or a group (CH₂)ₙ—X²⁹,

wherein:

X²⁹ denotes halogen, NO₂, —OR²⁰⁴, —COR²⁰⁴, —CO₂R²⁰⁴, —OCO₂R²⁰⁴, —CN, —CONR²⁰⁴OR²⁰⁵—CONR²⁰⁴R²⁰⁵, —SR²⁰⁴ , —S(O)R²⁰⁴, —S(O)₂R²⁰⁴, —NR²⁰⁴R²⁰⁵, —NHC(O)R²⁰⁴, —NHS(O)₂R²⁰⁴; Z⁵ denotes —CH₂—, —CH₂—CH₂—, —CH₂—CH₂—CH₂—, —CH₂—CH═CH—, —CH═CH—CH₂—, —CH₂—CO—, —CO—CH₂—, —NHCO—, —CONH—, —NHCH₂—, —CH₂NH—, —N═CH—, —NHCH—, —CH₂—CH₂—NH—, —CH═CH—, >N—R²⁰³, >C═O, >S(O)ₘ;

R²⁰⁴ and R²⁰⁵ independently of each other denote hydrogen, alkyl, aralkyl or aryl;

n is an integer from 0 to 6;

R²⁰⁶ is a straight-chained or branched C₁₋₄-alkyl group which may optionally be mono- or polysubstituted by halogen or alkoxy, or R²⁰⁶ denotes CF₃; and

m denotes an integer from 0 to 2;

with the proviso that A¹² does not represent O if R²⁰⁶ denotes CF₃;

and the pharmaceutically acceptable salts thereof.

Cox-2 selective inhibitors that are useful in the subject method and compositions can include the compounds that are described in U.S. Pat. Nos. 6,169,188, 6,020,343, 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); and U.S. Pat. No. 6,359,182 (C-nitroso compounds).

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

In an embodiment of the present method, a subject in need of prevention, treatment or amelioration of pain, inflammation, or inflammation-associated disorder is treated with a cyclooxygenase-2 selective inhibitor and a low-dose of enteric coated aspirin. In one embodiment, the subject is treated with a combination that includes an amount of enteric coated aspirin, which is a low-dose of aspirin, and an amount of a Cox-2 selective inhibitor, where the amount of the enteric coated aspirin and the amount of the Cox-2 selective inhibitor together provide a dosage or amount of the combination that is sufficient to constitute an effective amount of the combination. The effective amount can be a pain or inflammation suppressing treatment or prevention effective amount.

The Cox-2 selective inhibitor that is used in the subject method can be any cyclooxygenase-2 selective inhibitor that is described above. Likewise, the aspirin that is used in the subject method is enteric coated aspirin, as that compound is described herein.

In the subject method, the enteric coated aspirin can be used in any amount that is an effective amount. It is preferred, however, that the amount of enteric coated aspirin that is administered is within a range of about 40 mg/day to about 2,000 mg/day. It is more preferred that the amount of the enteric coated aspirin is within a range of about 40 mg/day to about 325 mg/day, an amount that is within a range of about 40 mg/day to about 80 mg/day, is even more preferred. In one embodiment of the present method, it is preferred that the aspirin is administered at a dosage rate that is below 75 mg/day, and a range of about 40 mg/day to below 75 mg/day is more preferred.

When the term “about” is used herein in relation to a dosage amount of aspirin, it is to be understood to mean an amount that is within ±3 mg. Thus, “about 40-80 mg/day” includes all dosages within 37 to 83 mg/day.

Another embodiment of the present invention includes a composition for the treatment, prevention, or inhibition or pain, inflammation, or inflammation-associated disorder comprising enteric coated aspirin and a cyclooxygenase-2 selective inhibitor or prodrug thereof. It is preferred that a dose of the composition constitutes an amount of enteric coated aspirin and an amount of a cyclooxygenase-2 selective inhibitor or a pharmaceutically acceptable salt or prodrug thereof which together constitute a pain or inflammation suppressing treatment or prevention effective amount. When the composition is combined with a pharmaceutically-acceptable excipient, a pharmaceutical composition can be formed, which comprises enteric coated aspirin; a cyclooxygenase-2 selective inhibitor or prodrug thereof; and the pharmaceutically-acceptable excipient.

In another embodiment, a kit can be produced that is suitable for use in the treatment, prevention or inhibition of pain, inflammation or inflammation-associated disorder. The kit comprises a first dosage form comprising enteric coated aspirin and a second dosage form comprising a cyclooxygenase-2 selective inhibitor or prodrug thereof, in quantities which comprise a therapeutically effective amount of the combination of the compounds for the treatment, prevention, or inhibition of pain, inflammation or inflammation-associated disorder.

In another embodiment of the present method for the prevention, treatment, or amelioration of pain, inflammation, or inflammation-related disorder in a subject that is in need of such prevention, treatment or amelioration, the method comprises administering to the subject a cyclooxygenase-2 selective inhibitor or prodrug thereof and a low-dose of aspirin, wherein the aspirin is administered at a dosage level of below 75 mg/day.

In the method just described, the cycloxygenase-2 selective inhibitor can be any one of the cyclooxygenase-2 selective inhibitors that is described above, or a prodrug thereof.

In another embodiment, the invention includes a composition comprising a cyclooxygenase-2 selective inhibitor or prodrug thereof and a low-dose of aspirin, wherein the aspirin is present in an amount of below 75 mg. When this combination is combined with a pharmaceutically-acceptable excipient, a pharmaceutical composition is formed which includes a cyclooxygenase-2 selective inhibitor and a low-dose of aspirin in combination with a pharmaceutically acceptable carrier, wherein the aspirin is present in an amount of below 75 mg.

A kit that is suitable for use in the treatment, prevention or inhibition of pain, inflammation or inflammation-associated disorder can be provided which includes a first dosage form comprising less than 75 mg of aspirin and a second dosage form comprising a cyclooxygenase-2 selective inhibitor or prodrug thereof, wherein the cyclooxygenase-2 selective inhibitor is present in a quantity which, along with the quantity of aspirin, comprises a therapeutically effective amount of the combination of the compounds for the treatment, prevention, or inhibition of pain, inflammation or inflammation-associated disorder.

In another embodiment, the invention includes a method for the prevention, treatment, or amelioration of pain, inflammation, or inflammation-related disorder in a subject that is in need of such prevention, treatment or amelioration, the method comprising administering to the subject a combination comprising cyclooxygenase-2 selective inhibitor and aspirin, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof.

A composition can also be provided that includes a cyclooxygenase-2 selective inhibitor and aspirin, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof. When a pharmaceutically-acceptable excipient is added to this composition, a pharmaceutical composition is formed which comprises a cyclooxygenase-2 selective inhibitor and aspirin in combination with a pharmaceutically acceptable carrier, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof.

The invention also includes a kit that is suitable for use in the treatment, prevention or inhibition of pain, inflammation or inflammation-associated disorder, the kit comprises a first dosage form comprising aspirin and a second dosage form comprising a cyclooxygenase-2 selective inhibitor or prodrug thereof, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof, and wherein the aspirin and the cyclooxygenase-2 selective inhibitor are present in quantities which comprise a therapeutically effective amount of the combination of the compounds for the treatment, prevention, or inhibition of pain, inflammation or inflammation-associated disorder.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or inhibition”, and both are intended to qualify the amount of each agent for use in the combination therapy which will achieve the goal of improvement in the severity of pain and inflammation and the frequency of incidence over treatment of each agent by itself, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711.

The frequency of dose will depend upon the half-life of the active components of the composition. If the active molecules have a short half life (e.g. from about 2 to 10 hours) it may be necessary to give one or more doses per day. Alternatively, if the active molecules have a long half-life (e.g. from about 2 to about 15 days) it may only be necessary to give a dosage once per day, per week, or even once every 1 or 2 months. A preferred dosage rate is to administer the dosage amounts described above to a subject once per day.

For the purposes of calculating and expressing a dosage rate, all dosages that are expressed herein are calculated on an average amount-per-day basis irrespective of the dosage rate. For example, one 325 mg dosage of aspirin taken once every two days would be expressed as a dosage rate of 162 mg/day. Similarly, the dosage rate of an ingredient where 50 mg is taken twice per day would be expressed as a dosage rate of 100 mg/day.

For purposes of calculation of dosage amounts, the weight of a normal adult human will be assumed to be 70 kg.

In the subject method, it is preferred that the amount of the cyclooxygenase-2 selective inhibitor or prodrug thereof is within a range of from about 0.01 to about 100 mg/day per kg of body weight of the subject. It is more preferred that the amount of the cyclooxygenase-2 selective inhibitor or prodrug thereof is within a range of from about 1 to about 20 mg/day per kg of body weight of the subject.

When the Cox-2 selective inhibitor comprises rofecoxib, it is preferred that the amount used is within a range of from about 0.15 to about 1.0 mg/day.kg, and even more preferably from about 0.18 to about 0.4 mg/day.kg.

When the Cox-2 selective inhibitor comprises etoricoxib, it is preferred that the amount used is within a range of from about 0.5 to about 5 mg/day.kg, and even more preferably from about 0.8 to about 4 mg/day.kg.

When the Cox-2 selective inhibitor comprises celecoxib, it is preferred that the amount used is within a range of from about 1 to about 10 mg/day.kg, even more preferably from about 1.4 to about 8.6 mg/day.kg, and yet more preferably from about 2 to about 3 mg/day.kg.

In the subject method, it is preferred that the weight ratio of the amount of enteric coated aspirin to the amount of cyclooxygenase-2 selective inhibitor or prodrug thereof that is administered to the subject is within a range of from about 0.006:1 to about 3,000:1. It is more preferred that the weight ratio is within a range of from about 0.03:1 to about 5:1, and even more preferred that the weight ratio is within a range of from about 0.03:1 to about 1:1.

The combination of aspirin and a Cox-2 selective inhibitor can be supplied in the form of the novel therapeutic compositions described above, which are believed to be within the scope of the present invention. The relative amounts of each component in the therapeutic composition may be varied and may be as described above. The aspirin and Cox-2 selective inhibitor can be provided in the therapeutic composition so that the preferred amounts of each of the components are supplied by a single dosage, a single injection or a single capsule for example, or, by up to four, or more, single dosage forms.

When the novel combination is supplied along with a pharmaceutically acceptable carrier, the pharmaceutical compositions that are described above can be formed. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the combination of the invention are the isomeric forms and tautomers and the pharmaceutically-acceptable salts of aspirin and cyclooxygenase-2 selective inhibitors. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The method and combination of the present invention are useful for, but not limited to, the treatment, prevention and/or amelioration of pain and/or inflammation in a subject, and for treatment of inflammation-associated disorders, such as for use as an analgesic in the treatment of pain and headaches, or as an antipyretic for the treatment of fever. For example, combinations of the invention would be useful to treat arthritis, including, but not limited to, rheumatoid arthritis, spondyloarthopathies, gouty arthritis, osteoarthritis, systemic lupus erythematosus and juvenile arthritis. Such combinations of the invention would be useful in the treatment of asthma, bronchitis, menstrual cramps, tendinitis, bursitis, connective tissue injuries or disorders, and skin related conditions such as psoriasis, eczema, burns and dermatitis.

Combinations of the invention also would be useful to treat gastrointestinal conditions such as inflammatory bowel disease, gastric ulcer, gastric varices, Crohn's disease, gastritis, irritable bowel syndrome and ulcerative colitis and for the prevention or treatment of cancer, such as colorectal cancer. Combinations of the invention would be useful in treating inflammation in diseases and conditions such as herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, and the like.

Compositions having the novel combination would also be useful in the treatment of ophthalmic diseases, such as retinitis, retinopathies, conjunctivitis, uveitis, ocular photophobia, and of acute injury to the eye tissue. The compositions would also be useful in the treatment of pulmonary inflammation, such as that associated with viral infections and cystic fibrosis. The compositions would also be useful for the treatment of certain central nervous system disorders such as cortical dementias including Alzheimer's disease. The combinations of the invention are also useful as anti-inflammatory agents, such as for the treatment of arthritis.

As used herein, the terms “pain, inflammation or inflammation-associated disorder”, and “cyclooxygenase-2 mediated disorder” are meant to include, without limitation, each of the symptoms or diseases that is mentioned above.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of pain and/or inflammation associated with, but not limited to, any of the diseases or disorders described herein. Besides being useful for human treatment, these combinations are also useful for treatment of mammals, including horses, dogs, cats, rats, mice, sheep, pigs, etc.

The term “amelioration” includes the improvement of symptoms caused by or associated with pain or inflammation, or the inflammation-related disorders described above.

The term “subject” for purposes of treatment includes any human or animal subject who is in need of the prevention of, or who has pain, inflammation and/or any one of the known inflammation-associated disorders. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

For methods of prevention, the subject is any human or animal subject, and preferably is a subject that is in need of prevention and/or treatment of pain, inflammation and/or an inflammation-associated disorder. The subject may be a human subject who is at risk for pain and/or inflammation, or for obtaining an inflammation-associated disorder, such as those described above. The subject may be at risk due to genetic predisposition, sedentary lifestyle, diet, exposure to disorder-causing agents, exposure to pathogenic agents and the like.

The subject pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrases “combination therapy”, “co-administration”, “administration with”, or “co-therapy”, in defining the use of aspirin and a cyclooxygenase-2 selective, is intended to embrace administration of each agent in a sequential manner in a regimen that will provide beneficial effects of the drug combination, and is intended as well to embrace co-administration of these agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from both of the constituent agents of the combination.

Although the combination of the present invention may include administration of an aspirin component and a cyclooxygenase-2 selective inhibitor component within an effective time of each respective component, it is preferable to administer both respective components contemporaneously, and more preferable to administer both respective components in a single delivery dose.

In particular, the combinations of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject combinations can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject combination can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### COMPARATIVE EXAMPLE 1

This example shows the preparation of celecoxib.

Step 1: Preparation of 1-(4-methylphenyl)-4,4,4-trifluorobutane-1,3-dione.

Following the disclosure provided in U.S. Pat. No. 5,760,068, 4′-Methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCl was added and the mixture extracted with 4×75 mL ethyl acetate. The extracts were dried over MgSO₄, filtered and concentrated to afford 8.47 g (94%) of a brown oil which was carried on without further purification.

Step 2: Preparation of 4-[5-(4-methylphenyl)-3-(trifluoromethyl)-1H-pyrazol -1-yl]benzenesulfonamide.

To the dione from Step 1 (4.14 g, 18.0 mmol) in 75 mL absolute ethanol, 4.26 g (19.0 mmol) 4-sulphonamidophenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157°-159° C.; and a calculated composition of C₁₇H₁₋₄N₃O₂SF₃; C, 53.54; H, 3.70; N, 11.02. The composition that was found by analysis was: C, 53.17; H, 3.81; N, 10.90.

### EXAMPLE 2

This illustrates the production of a composition containing celecoxib and enteric coated aspirin, and of a pharmaceutical composition containing the combination.

Celecoxib can be prepared as described in Comparative Example 1, or it can be obtained under the trade name CELEBREX® from Pharmacia Corporation, Peapack, N.J. Enteric coated aspirin can be obtained from several commercial suppliers. One example is ECOTRIN®, a form of enteric coated aspirin available from GlaxoSmithKlein, Greenford, Middlesex, UK.

A therapeutic composition of the present invention can be formed by intermixing enteric coated aspirin (80 g, available as ECOTRIN® from GlaxoSmithKlein, Greenford, Middlesex, UK), and 4-[5-(4-methylphenyl)-3-(trifluoromethyl)-1H-pyrazol-1-yl]benzenesulfonamide (200 g, as produced in Comparative Example 1, or as available from Pharmacia Corporation, Peapack, N.J.), in a laboratory mill or mixing device suitable for intimate mixing of powders without substantial generation of shear or temperature sufficient to degrade either of the two compounds. After mixing, the combination of celecoxib and enteric coated aspirin form a therapeutic composition that is sufficient for the production of about 1000 human single dose units. Each single dose unit contains about 80 mg of enteric coated aspirin and about 200 mg of celecoxib.

If desirable, a solid carrier and other materials may be intermixed with the therapeutic composition to form a pharmaceutical composition and the resulting pharmaceutical composition may be formed into capsules for human consumption, for example, by conventional capsule-forming equipment, where each capsule contains 80 mg of enteric coated aspirin and 200 mg celecoxib.

Alternatively, the enteric coated aspirin and the celecoxib may be suspended in a liquid carrier, such as, for example, normal saline solution, to form a pharmaceutical composition suitable for human consumption. A single dosage of the liquid pharmaceutical composition for human use would be a volume sufficient to provide 80 mg of enteric coated aspirin and 200 mg of celecoxib.

Therapeutic and pharmaceutical compositions comprising a combination of any of the cyclooxygenase-2 selective inhibitors and any enteric coated aspirin that are described above can be formed by similar methods.

### EXAMPLE 3

This illustrates the evaluation of the biological efficacy of a therapeutic composition of enteric coated aspirin and celecoxib for the alleviation of pain and inflammation.

A therapeutic composition containing enteric coated aspirin and celecoxib is prepared as described in Example 2. The biological efficacy of the composition is determined by a rat carrageenan foot pad edema test and by a rat carrageenan-induced analgesia test.

Rat Carrageenan Foot Pad Edema Test:

The carrageenan foot edema test is performed with materials, reagents and procedures essentially as described by Winter, et al., (Proc. Soc. Exp. Biol. Med., 111, 544 (1962)). Male Sprague-Dawley rats are selected in each group so that the average body weight is as close as possible. Rats are fasted with free access to water for over sixteen hours prior to the test. The rats are dosed orally (1 mL) with the enteric coated aspirin and celecoxib composition that is described in Example 2 suspended in a carrier vehicle containing 0.5% methylcellulose and 0.025% surfactant, or with only the carrier vehicle alone. One hour later, a subplantar injection of 0.1 mL of 1% solution of carrageenan/sterile 0.9% saline is administered to one foot and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDS, in Non-steroidal Anti-Inflammatory Drugs, (J. Lombardino, ed. 1985)). The percent inhibition shows the percent decrease from control paw volume determined in this procedure. It is believed that the data would show that the combination of enteric coated aspirin and celecoxib provides effective anti-inflammatory activity.

Rat Carrageenan-induced Analgesia Test:

The analgesia test using rat carrageenan is performed with materials, reagents and procedures essentially as described by Hargreaves, et al., (Pain, 32, 77 (1988)). Male Sprague-Dawley rats are treated as previously described for the Carrageenan Foot Pad Edema test. Three hours after the injection of the carrageenan, the rats are placed in a special PLEXIGLAS® container with a transparent floor having a high intensity lamp as a radiant heat source, positionable under the floor. After an initial twenty-minute period, thermal stimulation is begun on either the injected foot or on the contralateral uninjected foot. A photoelectric cell will turn off the lamp and timer when the light is interrupted by paw withdrawal. The time until the rat withdraws its foot is then measured. The withdrawal latency in seconds is determined for the control and drug-treated groups, and percent inhibition of the hyperalgesic foot withdrawal is determined. It is believed that results would show that a combination of enteric coated aspirin and celecoxib provides effective analgesic activity.

### EXAMPLE 4

This illustrates the biological efficacy of a therapeutic composition of enteric coated aspirin and celecoxib for the treatment of collagen-induced arthritis in mice.

A therapeutic composition containing enteric coated aspirin and celecoxib is prepared as described in Example 2. The biological efficacy of the composition is determined by induction and assessment of collagen-induced arthritis in mice.

Arthritis is induced in 8-12 week old male DBA/1 mice by injection of 50 μg of chick-type II collagen (CII) in complete Freunds adjuvant (Sigma) on day 0 at the base of the tail as described in [J. Stuart, Annual Rev. Immunol., 2, 199 (1984)]. Compounds are prepared as a suspension in 0.5% methylcellulose (Sigma, St. Louis, Mo.), and 0.025% Tween 20 (Sigma). The cyclooxygenase-2 inhibitor (celecoxib, as described in Comparative Example 1), and enteric coated aspirin (available under the trade name ECOTRIN® from GlaxoSmithKlein) are administered alone or in combination as a therapeutic composition as described in Example 2. The compounds are administered in non-arthritic animals by gavage in a volume of 0.1 ml beginning on day 20 post collagen injection and continuing daily until final evaluation on day 55. Animals are boosted on day 21 with 50 μg of collagen (CII) in incomplete Freunds adjuvant. The animals are subsequently evaluated several times each week for incidence and severity of arthritis until day 56. Any animal with paw redness or swelling is counted as arthritic. Scoring of severity is carried out using a score of 0-3 for each paw (maximal score of 12/mouse) as described in P. Wooley, et al., Trans. Proc., 15, 180 (1983). The animals are measured for incidence of arthritis and severity in the animals where arthritis was observed. The incidence of arthritis is determined at a gross level by observing the swelling or redness in the paw or digits. Severity is measured with the following guidelines. Briefly, animals displaying four normal paws, i.e., no redness or swelling are scored 0. Any redness or swelling of digits or the paw are scored as 1. Gross swelling of the whole paw or deformity is scored as 2. Ankylosis of joints is scored as 3.

Histological Examination of Paws:

In order to verify the gross determination of a non-arthritic animal, a histological examination can be performed. Paws from animals sacrificed at the end of the experiment are removed, fixed and decalcified as previously described [R. Jonsson, J. Immunol. Methods, 88,109 (1986)]. Samples are paraffin embedded, sectioned, and stained with hematoxylin and eosin by standard methods. Stained sections are examined for cellular infiltrates, synovial hyperplasia, and bone and cartilage erosion.

It is believed that results will show that the combination of a cyclooxygenase-2 selective inhibitor with the enteric coated aspirin is an efficacious treatment for collagen-induced arthritis in mice.

It is believed that Examples 3 and 4 can be repeated with compositions comprising enteric coated aspirin, or regular aspirin where appropriate, in combination with any of the cyclooxygenase-2 selective inhibitors that are described herein, with the results showing that the combination provides effective anti-inflammatory activity, effective analgesic activity, and is an efficacious treatment of collagen-induced arthritis in mice.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. A method for the prevention, treatment, or amelioration of pain, inflammation, or inflammation-related disorder in a subject that is in need of such prevention, treatment or amelioration, the method comprising administering to the subject a cyclooxygenase-2 selective inhibitor or prodrug thereof and enteric coated aspirin.

2. The method according to claim 1, wherein the method comprises administering to a subject that is in need of such prevention, treatment or amelioration a combination comprising an amount of a cycloxygenase-2 selective inhibitor or prodrug thereof and an amount of enteric coated aspirin wherein the amount of the cyclooxygenase-2 selective inhibitor and the amount of enteric coated aspirin comprise an effective amount of the combination.

3. The method according to claim 2, wherein the effective amount of the combination is a therapeutically effective amount.

4. The method according to claim 1, wherein the cyclooxygenase-2 selective inhibitor or prodrug thereof has a cyclooxygenase-2 IC₅₀ of less than about 0.2 μmol/L.

5. The method according to claim 4, wherein the cyclooxygenase-2 selective inhibitor or prodrug thereof has a cyclooxygenase-1 IC₅₀ of at least about 1 μmol/L.

6. The method according to claim 5, wherein the cyclooxygenase-2 selective inhibitor or prodrug thereof has a cyclooxygenase-1 IC₅₀ of at least about 10 μmol/L.

7. The method according to claim 1, wherein the cyclooxygenase-2 selective inhibitor is selected from the group consisting of celecoxib, valdecoxib, deracoxib, rofecoxib, etoricoxib, parecoxib, lumiracoxib, SD-8381, ABT-963, BMS-347070, and NS-398.

8. The method according to claim 7, wherein the cyclooxygenase-2 selective inhibitor is selected from the group consisting of celecoxib, valdecoxib, deracoxib, rofecoxib, etoricoxib, parecoxib, and lumiracoxib.

9. The method according to claim 8, wherein the cyclooxygenase-2 selective inhibitor is selected from the group consisting of celecoxib, valdecoxib, and parecoxib.

10. The method according to claim 1, wherein the cyclooxygenase-2 selective inhibitor comprises celecoxib.

11. The method of claim 1, wherein the amount of the enteric coated aspirin is within a range of about 40 mg/day to about 2,000 mg/day.

12. The method of claim 11, wherein the amount of the enteric coated aspirin is within a range of about 40 mg/day to about 325 mg/day.

13. The method of claim 12, wherein the amount of the enteric coated aspirin is within a range of about 40 mg/day to about 80 mg/day.

14. The method of claim 1, wherein the amount of the enteric coated aspirin is within a range of about 40 mg/day to below 75 mg/day.

15. The method according to claim 1, wherein the amount of the cyclooxygenase-2 selective inhibitor or prodrug thereof is within a range of from about 0.01 to about 100 mg/day per kg of body weight of the subject.

16. The method according to claim 15, wherein the amount of the cyclooxygenase-2 selective inhibitor or prodrug thereof is within a range of from about 1 to about 20 mg/day per kg of body weight of the subject.

17. The method according to claim 1, wherein the weight ratio of the amount of enteric coated aspirin to the amount of cyclooxygenase-2 selective inhibitor or prodrug thereof that is administered to the subject is within a range of from about 0.006:1 to about 3,000:1.

18. The method according to claim 17, wherein the weight ratio of the amount of enteric coated aspirin to the amount of cyclooxygenase-2 selective inhibitor or prodrug thereof that is administered to the subject is within a range of from about 0.03:1 to about 5:1.

19. The method according to claim 18, wherein the weight ratio of the amount of enteric coated aspirin to the amount of cyclooxygenase-2 selective inhibitor or prodrug thereof that is administered to the subject is within a range of from about 0.03:1 to about 1:1.

20. The method according to claim 1, wherein the pain, inflammation or inflammation associated disorder is selected from the group consisting of headache, fever, arthritis, rheumatoid arthritis, spondyloarthopathies, gouty arthritis, osteoarthritis, systemic lupus erythematosus, juvenile arthritis, asthma, bronchitis, menstrual cramps, tendinitis, bursitis, connective tissue injuries or disorders, skin related conditions, psoriasis, eczema, burns, dermatitis, gastrointestinal conditions, inflammatory bowel disease, gastric ulcer, gastric varices, Crohn's disease, gastritis, irritable bowel syndrome, ulcerative colitis, cancer, colorectal cancer, herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, ophthalmic diseases, retinitis, retinopathies, conjunctivitis, uveitis, ocular photophobia, acute injury to the eye tissue, pulmonary inflammation, nervous system disorders, cortical dementias, and Alzheimer's disease.

21. The method according to claim 1, wherein the pain, inflammation or inflammation associated disorder is an opthalmic disease or opthalmic injury.

22. The method according to claim 21, wherein the opthalmic disease or opthalmic injury is selected from the group consisting of retinitis, retinopathies, conjunctivitis, uveitis, ocular photophobia, acute injury to the eye tissue,

23. The method according to claim 20, wherein the pain, inflammation or inflammation associated disorder is arthritis.

24. The method according to claim 23 wherein the arthritis is osteoarthritis.

25. The method according to claim 23 wherein the arthritis is rheumatoid arthritis.

26. The method according to claim 1, wherein the subject is an animal.

27. The method according to claim 26, wherein the subject is a human.

28. The method according to claim 1, wherein the treating step comprises administering enteric coated aspirin and a cycloxoygenase-2 selective inhibitor to the subject enterally or parenterally in one or more dose per day.

29. The method according to claim 28, wherein the enteric coated aspirin and the cycoloxygenase-2 selective inhibitor are administered to the subject substantially simultaneously.

30. The method according to claim 28, wherein the enteric coated aspirin and the cycoloxygenase-2 selective inhibitor are administered sequentially.

31. A composition for the treatment, prevention, or inhibition or pain, inflammation, or inflammation-associated disorder comprising enteric coated aspirin and a cyclooxygenase-2 selective inhibitor or prodrug thereof.

32. The composition according to claim 31, wherein the composition is useful for treating a subject in need of treatment, prevention, or inhibition of pain, inflammation, or an inflammation-associated disorder, and wherein a dose of the composition constitutes an amount of enteric coated aspirin and an amount of a cyclooxygenase-2 selective inhibitor or a pharmaceutically acceptable salt or prodrug thereof which together constitute a pain or inflammation suppressing treatment or prevention effective amount.

33. A pharmaceutical composition comprising enteric coated aspirin; a cyclooxygenase-2 selective inhibitor or prodrug thereof; and a pharmaceutically-acceptable excipient.

34. A kit that is suitable for use in the treatment, prevention or inhibition of pain, inflammation or inflammation-associated disorder, the kit comprises a first dosage form comprising enteric coated aspirin and a second dosage form comprising a cyclooxygenase-2 selective inhibitor or prodrug thereof, in quantities which comprise a therapeutically effective amount of the combination of the compounds for the treatment, prevention, or inhibition of pain, inflammation or inflammation-associated disorder.

35. A method for the prevention, treatment, or amelioration of pain, inflammation, or inflammation-related disorder in a subject that is in need of such prevention, treatment or amelioration, the method comprising administering to the subject a cyclooxygenase-2 selective inhibitor or prodrug thereof and a low-dose of aspirin, wherein the aspirin is administered at a dosage level of below 75 mg/day.

36. The method according to claim 35, wherein the cycloxygenase-2 selective inhibitor or prodrug thereof is selected from the group consisting of celecoxib, valdecoxib, deracoxib, rofecoxib, etoricoxib, parecoxib, lumiracoxib, SD-8381, ABT-963, BMS-347070, and NS-398.

37. The method according to claim 36, wherein the cyclooxygenase-2 selective inhibitor is selected from the group consisting of celecoxib, valdecoxib, deracoxib, rofecoxib, etoricoxib, parecoxib, and lumiracoxib.

38. The method according to claim 37, wherein the cyclooxygenase-2 selective inhibitor is selected from the group consisting of celecoxib, valdecoxib, and parecoxib.

39. A composition comprising a cyclooxygenase-2 selective inhibitor or prodrug thereof and a low-dose of aspirin, wherein the aspirin is present in an amount of below 75 mg.

40. A pharmaceutical composition comprising a cyclooxygenase-2 selective inhibitor and a low-dose of aspirin in combination with a pharmaceutically acceptable carrier, wherein the aspirin is present in an amount of below 75 mg.

41. A kit that is suitable for use in the treatment, prevention or inhibition of pain, inflammation or inflammation-associated disorder, the kit comprises a first dosage form comprising less than 75 mg of aspirin and a second dosage form comprising a cyclooxygenase-2 selective inhibitor or prodrug thereof, wherein the cyclooxygenase-2 selective inhibitor is present in a quantity which, along with the quantity of aspirin, comprises a therapeutically effective amount of the combination of the compounds for the treatment, prevention, or inhibition of pain, inflammation or inflammation-associated disorder.

42. A method for the prevention, treatment, or amelioration of pain, inflammation, or inflammation-related disorder in a subject that is in need of such prevention, treatment or amelioration, the method comprising administering to the subject a combination comprising cyclooxygenase-2 selective inhibitor and aspirin, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof.

43. The method according to claim 42, wherein the weight ratio of the amount of aspirin to the amount of cyclooxygenase-2 selective inhibitor or prodrug thereof that is administered to the subject is within a range of from about 0.03:1 to about 5:1.

44. The method according to claim 43, wherein the weight ratio of the amount of aspirin to the amount of cyclooxygenase-2 selective inhibitor or prodrug thereof that is administered to the subject is within a range of from about 0.03:1 to about 1:1.

45. A composition comprising a cyclooxygenase-2 selective inhibitor and aspirin, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof.

46. A pharmaceutical composition comprising a cyclooxygenase-2 selective inhibitor and aspirin in combination with a pharmaceutically acceptable carrier, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof.

47. A kit that is suitable for use in the treatment, prevention or inhibition of pain, inflammation or inflammation-associated disorder, the kit comprises a first dosage form comprising aspirin and a second dosage form comprising a cyclooxygenase-2 selective inhibitor or prodrug thereof, wherein the cyclooxygenase-2 selective inhibitor is selected from the group consisting of BMS-347070, S-33516, CS-502, darbufelone, LAS 34475, LAS 34556, L-745337, SD-8381, RWJ-63556, L-784512, COX-189, ABT-963, valdecoxib, and any pharmaceutical salt or prodrug thereof, and wherein the aspirin and the cyclooxygenase-2 selective inhibitor are present in quantities which comprise a therapeutically effective amount of the combination of the compounds for the treatment, prevention, or inhibition of pain, inflammation or inflammation-associated disorder.
